(19) United States
(12) Patent Application Publication
(10) Pub. No.: US 2022/0112473 A1
(43) Pub. Date: Apr. 14, 2022

(54) ENGINEERED PROTEINS AND METHODS OF USE THEREOF

(71) Applicant: Pairwise Plants Services, Inc., Durham, NC (US)

(72) Inventors: Sharon Leigh Guffy, Durham, NC (US); Joseph Matthew Watts, Cary, NC (US)

(21) Appl. No.: 17/459,166

(22) Filed: Aug. 27, 2021

Related U.S. Application Data
(60) Provisional application No. 63/071,734, filed on Aug. 28, 2020.

(51) Int. Cl. C12N 9/22; C12N 15/11

(52) U.S. Cl. CPC C12N 9/22; C12N 2310/20; C12N 15/111

(57) ABSTRACT

Described herein are engineered proteins and methods of use of such proteins. Also described herein are complexes, compositions, and systems including engineered proteins of the present invention, each of which may be used for modifying or editing a target nucleic acid. An engineered protein of the present invention may be an enzyme and/or may be an RNA-guided DNA-binding protein.

### STATEMENT REGARDING ELECTRONIC FILING OF A SEQUENCE LISTING

A Sequence Listing in ASCII text format, submitted under 37 C.F.R. § 1.821, entitled 1499-37_ST25, 922,214 bytes in size, generated on Aug. 27, 2021, and filed via EFS-Web, is provided in lieu of a paper copy. This Sequence Listing is hereby incorporated herein by reference into the specification for its disclosures.

### FIELD

This invention relates to engineered proteins (e.g., engineered enzymes) and to methods of use of such proteins. The invention further relates to compositions and systems for modifying or editing a target nucleic acid.

### BACKGROUND OF THE INVENTION

Type II CRISPR endonucleases, including the widely used SpCas9, share a common mechanism for DNA cleavage. Enzymes in this family contain two nuclease domains (HNH and RuvC), each of which cleaves a single DNA strand. When the Cas9-sgRNA complex (or Cas9-crRNA-trRNA complex) binds its target DNA sequence, the target DNA strand binds to the RNA spacer sequence while the nontarget DNA strand forms a single-stranded loop. The HNH domain of Cas9 cleaves the target DNA strand, and the RuvC domain cleaves the nontarget strand (FIG. 1). As illustrated in FIG. 1, for Type II CRISPR endonucleases (e.g., Cas9), the target and nontarget DNA strands are cleaved simultaneously by the HNH and RuvC domains, respectively, forming blunt-ended double strand breaks.

Unlike the Type II CRISPR endonucleases, the Type V CRISPR endonucleases (such as Cas12a) have only a single nuclease domain that sequentially cleaves both DNA strands beginning with the nontarget strand. As illustrated in FIG. 2, for Type V endonucleases (e.g., Cas12a), the RuvC domain cleaves the nontarget and target DNA strands sequentially, resulting in staggered double-strand breaks.

Although Type II and Type V CRISPR endonucleases perform similar functions, their mechanisms and structures are highly divergent. The two different types are thought to have evolved from different precursors, and only the RuvC domain shares any significant sequence or structural homology across the two types. Type V CRISPR endonucleases lack the HNH domain responsible for target strand cleavage in Type II enzymes. Instead, the RuvC domain in Type V CRISPR endonucleases cleaves both DNA strands sequentially (FIG. 2) beginning with the nontarget strand. Therefore, mutating the catalytic residue of the RuvC domain prevents all nuclease activity and produces a deactivated enzyme rather than producing a target strand nickase. A nontarget strand nickase mutation has been identified in Cas12a; however, this mutation is outside the RuvC domain and is thought to function by reducing the overall catalytic efficiency of the enzyme. No Type V CRISPR target strand nickase exists, and, in view of the differences in structure and mechanism of action for Type V CRISPR endonucleases compared to Type II CRISPR endonucleases, there is no clear way of producing one.

### SUMMARY OF THE INVENTION

A first aspect of the present invention is directed to an engineered protein comprising at least two different polypeptides, wherein one of the at least two different polypeptides is a first CRISPR-Cas effector polypeptide that is a first portion of a first Type V CRISPR-Cas effector protein and the first CRISPR-Cas effector polypeptide is devoid of a nuclease domain; and wherein another of the least two different polypeptides is a heterologous polypeptide that is heterologous to the Type V CRISPR-Cas effector protein and is not a portion of a Type V CRISPR-Cas effector protein.

Another aspect of the present invention is directed to an engineered protein comprising: a first nuclease domain, wherein the first nuclease domain is a target strand nickase domain or a portion thereof, wherein the first nuclease domain is not a Type V nuclease domain or a portion thereof; and a first CRISPR-Cas effector polypeptide that is a portion of a Type V CRISPR-Cas effector protein. In some embodiments, the first nuclease domain is a target strand specific nickase domain or a target and nontarget strand nickase domain.

A further aspect of the present invention is directed to an engineered protein comprising: a first polypeptide that is a first portion of a first Type V CRISPR-Cas effector protein; a second polypeptide that is a second portion of the first Type V CRISPR-Cas effector protein; and a heterologous polypeptide that is heterologous to the first Type V CRISPR-Cas effector protein, wherein the heterologous polypeptide is between the first and second polypeptides and the heterologous polypeptide is positioned in the engineered protein in a location that corresponds to an interdomain linker region of the first Type V CRISPR-Cas effector protein.

An additional aspect of the present invention is directed to an engineered protein comprising an amino acid sequence having at least 70%, 75%, 80%, 85%, 90%, 95%, 96%, 97%, 98%, 99%, or more sequence identity to any one of SEQ ID NOs:2-17 or 125-132, optionally wherein the engineered protein comprises an amino acid sequence of any one of SEQ ID NOs:2-17 or 125-132.

A further aspect of the present invention is directed to a composition (e.g., a base editing composition) or system comprising: an engineered protein as described herein; a guide nucleic acid (e.g., a guide RNA), and optionally a deaminase, optionally wherein the engineered protein, guide nucleic acid, and optionally deaminase form a complex or are comprised in a complex.

Another aspect of the present invention is directed to a complex comprising: an engineered protein as described herein; a guide nucleic acid (e.g., a guide RNA); and optionally a deaminase.

An additional aspect of the present invention is directed to a nucleic acid molecule comprising a nucleotide sequence encoding an engineered protein as described herein.

Another aspect of the present invention is directed to a method of modifying a target nucleic acid, the method comprising: contacting the target nucleic acid with: an engineered protein as described herein, and a guide nucleic acid (e.g., a guide RNA), optionally wherein the engineered protein and the guide nucleic acid form a complex or are comprised in a complex, thereby modifying the target nucleic acid.

A further aspect of the present invention is directed to a method of increasing the efficiency of modifying a target nucleic acid, the method comprising: contacting the target nucleic acid with: an engineered protein as described herein, and a guide nucleic acid (e.g., a guide RNA), optionally wherein the engineered protein and the guide nucleic acid form a complex or are comprised in a complex, thereby modifying the target nucleic acid.

The invention further provides expression cassettes and/or vectors comprising a nucleic acid construct of the present invention, and cells comprising a polypeptide, fusion protein and/or nucleic acid construct of the present invention. Additionally, the invention provides kits comprising a nucleic acid construct of the present invention and expression cassettes, vectors and/or cells comprising the same.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is an illustration depicting the mechanism of action for Type II CRISPR endonucleases.

FIG. 2 is an illustration depicting the mechanism of action for Type V CRISPR endonucleases.

FIG. 3 is the crystal structure of SpCas9 (PDB ID 4UN3) bound to a single guide RNA (sgRNA) and target DNA. Domains shown are as follows: RuvC, bridge helix, Rec1, Rec2, HNH, and PAM-interacting.

FIG. 4 is a diagram of Cas12a domains viewed facing the Rec lobe. From this view, a portion of the crRNA/target DNA duplex is visibly exposed to the surface of Cas12a.

FIG. 5 is an overlay of the HNH domain from SpCas9 onto the candidate insertion site in LbCas12a.

FIG. 6 is an illustration depicting the soluble fraction lysed Escherichia coli expressing HNH-3287, 3288, 3289, 3290, 3296, 3297, 3298, and 3299.

FIG. 7 is an illustration depicting the nicking activity of purified HNH-3287, 3288, 3289, 3290, 3296, 3297, and 3298.

FIG. 8 is an image of a gel that indicates that nickases according to some embodiments of the present invention were solubly expressed in E. coli.

FIG. 9 is an image of a gel that indicates that nickases according to some embodiments of the present invention can nick a DNA substrate.

FIG. 10 is an image of a gel that indicates that nickases according to some embodiments of the present invention can be RNA-dependent.

FIG. 11 is an image of a gel that indicates that nickases according to some embodiments of the present invention can act as a DNA nickase.

FIG. 12 is an illustration showing a labeled target strand.

FIG. 13 is an illustration showing a labeled non-target strand.

FIG. 14 is an image of a gel including samples incubated with a labeled target strand.

FIG. 15 is an image of a gel including samples incubated with a labeled non-target strand.

FIG. 16 is an image of the entire gel showing the lanes of FIG. 14 and FIG. 15 along with the lanes for the controls.

FIG. 17 is an illustration showing editing efficiencies for respective enzyme pairs according to some embodiments of the present invention.

FIGS. 18-21 are graphs showing the percentage of C to T editing for various target regions corresponding to the respective spacers: FANCF spacer 1 (FIG. 18), FANCF spacer 2 (FIG. 19), AAVS1 spacer 1 (FIG. 20), and AAVS1 spacer 2 (FIG. 21).

FIGS. 22-23 are graphs showing the percentage of A to G editing for various target regions corresponding to the respective spacers: RNF2 spacer 1 (FIG. 22) and RNF2 spacer 2 (FIG. 23).

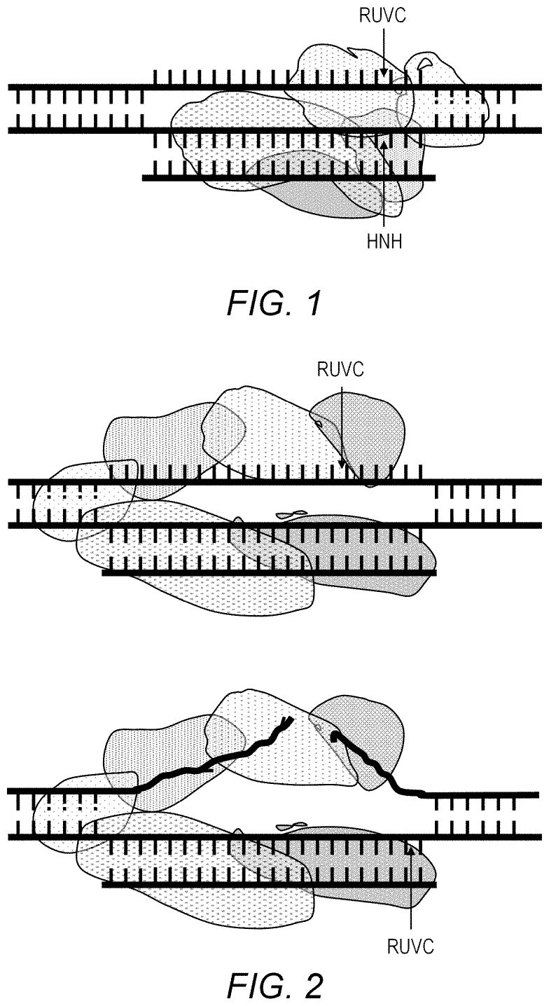

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even 0.1% of the specified value as well as the specified value. For example, “about X” where X is the measurable value, is meant to include X as well as variations of 10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “heterologous nucleotide sequence” or a “recombinant nucleotide sequence” is a nucleotide sequence not naturally associated with a host cell into which it is introduced, including non-naturally occurring multiple copies of a naturally occurring nucleotide sequence.

A “native” or “wild-type” nucleic acid, nucleotide sequence, polypeptide or amino acid sequence refers to a naturally occurring or endogenous nucleic acid, nucleotide sequence, polypeptide or amino acid sequence. Thus, for example, a “wild-type mRNA” is an mRNA that is naturally occurring in or endogenous to the reference organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “recombinant nucleic acid,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions).

A polynucleotide, gene, or polypeptide may be “isolated” by which is meant a nucleic acid or polypeptide that is substantially or essentially free from components normally found in association with the nucleic acid or polypeptide, respectively, in its natural state. In some embodiments, such components include other cellular material, culture medium from recombinant production, and/or various chemicals used in chemically synthesizing the nucleic acid or polypeptide.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., “substantially complementary,” such as about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence or polypeptide (including a domain) will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of a nucleotide sequence or polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide. Such a nucleic acid fragment or portion according to the invention may be, where appropriate, included in a larger polynucleotide of which it is a constituent. As an example, a repeat sequence of guide nucleic acid of this invention may comprise a portion of a wild-type CRISPR-Cas repeat sequence (e.g., a wild-type Type V CRISPR Cas repeat, e.g., a repeat from the CRISPR Cas system that includes, but is not limited to, Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c, and the like).

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptides of this invention. “Orthologous” and “orthologs” as used herein, refers to homologous nucleotide sequences and/or amino acid sequences in different species that arose from a common ancestral gene during speciation. A homologue or ortholog of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer).

Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention can be codon optimized for expression. In some embodiments, a polynucleotide, nucleic acid construct, expression cassette, and/or vector of the present invention (e.g., that comprises/encodes an engineered protein, a nucleic acid binding domain (e.g., a DNA binding domain such as a sequence-specific DNA binding domain from a polynucleotide-guided endonuclease, a zinc finger nuclease, a transcription activator-like effector nuclease (TALEN), an Argonaute protein, and/or a CRISPR-Cas effector protein), a guide nucleic acid, a cytosine deaminase and/or adenine deaminase) may be codon optimized for expression in an organism (e.g., an animal, a plant, a fungus, an archaeon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors but which have not been codon optimized.

In any of the embodiments described herein, a polynucleotide or nucleic acid construct of the invention may be operatively associated with a variety of promoters and/or other regulatory elements for expression in an organism or cell thereof (e.g., a plant and/or a cell of a plant). Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” or “fused” in reference to polypeptides, refers to the attachment of one polypeptide to another. A polypeptide may be linked or fused to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two domains of a fusion protein, such as, for example, a CRISPR-Cas effector protein and a peptide tag and/or a polypeptide of interest. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker. In some embodiments, the peptide linker has one of the amino acid sequences of SEQ ID NOs:18-47. In some embodiments, the peptide linker may comprise an amino acid sequence of (GGS)ₙ, GS, SG, GSSG (SEQ ID NO:175), S(GGS)ₙ(SEQ ID NO:42), SGGS (SEQ ID NO:43), or (GGGGS)n (SEQ ID NO:44), wherein n is an integer of 1-20 (e.g., 1, 2, 3, 4, 5, 6, 7, 8, 9, 10, 11, 12, 13, 14, 15, 16, 17, 18, 19, or 20). In some embodiments, the peptide linker may comprise the amino acid sequence: SGGSGGSGGS (SEQ ID NO:45). In some embodiments, the peptide linker may comprise the amino acid sequence: SGSETPGTSESATPES (SEQ ID NO:46), also referred to as the XTEN linker. In some embodiments, the peptide linker may comprise the amino acid sequence: SGGSSGGSSGSETPGTSESATPESSGGSSGGS (SEQ ID NO:47), also referred to as the GS-XTEN-GS linker.

As used herein, the term “linked,” or “fused” in reference to polynucleotides, refers to the attachment of one polynucleotide to another polynucleotide. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NO:48 or SEQ ID NO:49).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and Arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the European patent publication EP0342926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, incorporated by reference herein for its disclosure of promoters. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); European patent EP 0452269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA2-S promoter from Arabidopsis (U.S. Pat. No. 7,141,424), and/or the ZmC5 promoter from maize (International PCT Publication No. WO 1999/042587).

Additional examples of plant tissue-specific/tissue preferred promoters include, but are not limited to, the root hair-specific cis-elements (RHEs) (KIM ET AL. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983)Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992)Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987)Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

An “editing system” as used herein refers to any site-specific (e.g., sequence-specific) nucleic acid editing system now known or later developed, which system can introduce a modification (e.g., a mutation) in a nucleic acid in target specific manner. For example, an editing system (e.g., a site- and/or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which may comprise one or more polypeptide(s) and/or one or more polynucleotide(s) that when present and/or expressed together (e.g., as a system) in a composition and/or cell can modify (e.g., mutate) a target nucleic acid in a sequence specific manner. In some embodiments, an editing system (e.g., a site- and/or sequence-specific editing system) can comprise one or more polynucleotide(s) and/or one or more polypeptide(s), including but not limited to a nucleic acid binding domain (e.g., a DNA binding domain), a nuclease, another polypeptide, and/or a polynucleotide. In some embodiments, a CRISPR-Cas editing system is provided and/or is used that comprises an engineered protein of the present invention.

In some embodiments, an editing system comprises one or more sequence-specific nucleic acid binding polypeptide(s) (e.g., a DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system comprises one or more cleavage polypeptide(s) (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN).

A “nucleic acid binding domain” as used herein refers to a polypeptide or domain that binds or is capable of binding a nucleic acid (e.g., a target nucleic acid). A DNA binding domain is an example nucleic acid binding domain and may be a site- and/or sequence-specific nucleic acid binding domain. In some embodiments, a nucleic acid binding domain may be a sequence-specific nucleic acid binding domain such as, but not limited to, a sequence-specific binding domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding domain comprises a cleavage domain (e.g., a nuclease domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding domain is a polypeptide that can associate (e.g., form a complex) with one or more nucleic acid molecule(s) (e.g., form a complex with a guide nucleic acid as described herein) that can direct or guide the nucleic acid binding domain to a specific target nucleotide sequence (e.g., a gene locus of a genome) that is complementary to the one or more nucleic acid molecule(s) (or a portion or region thereof), thereby causing the nucleic acid binding domain to bind to the nucleotide sequence at the specific target site. In some embodiments, the nucleic acid binding domain is a CRISPR-Cas effector protein as described herein.

In some embodiments, an editing system comprises or is a ribonucleoprotein such as an assembled ribonucleoprotein complex (e.g., a ribonucleoprotein that comprises a CRISPR-Cas effector protein, a guide nucleic acid, and optionally a deaminase). In some embodiments, a ribonucleoprotein of an editing system may be assembled together (e.g., a pre-assembled ribonucleoprotein including a CRISPR-Cas effector protein, a guide nucleic acid, and optionally a deaminase) such as when contacted to a target nucleic acid or when introduced into a cell (e.g., a plant cell). In some embodiments, a ribonucleoprotein of an editing system may assemble into a complex (e.g., a covalently and/or non-covalently bound complex) while a portion of the ribonucleoprotein is contacting a target nucleic acid and/or may assemble after and/or during introduction into a plant cell. In some embodiments, an editing system may be assembled (e.g., into a covalently and/or non-covalently bound complex) when introduced into a plant cell. In some embodiments, a ribonucleoprotein may comprise an engineered protein, a guide nucleic acid, and optionally a deaminase.

The terms “transgene” or “transgenic” as used herein refer to at least one nucleic acid sequence that is taken from the genome of one organism or produced synthetically, and which is then introduced into a host cell (e.g., a plant cell) or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation into the genome and thus without inheritance of the respective at least one molecule introduced into the genome of a cell. The term “transgene-free” refers to a condition in which a transgene is not present or found in the genome of a host cell or tissue or organism of interest.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., a polynucleotide encoding an engineered protein, a polynucleotide encoding a cytosine deaminase, a polynucleotide encoding an adenine deaminase, a polynucleotide encoding a deaminase fusion protein, a polynucleotide encoding a peptide tag, a polynucleotide encoding an affinity polypeptide, a polynucleotide encoding a glycosylase, and/or a polynucleotide comprising a guide nucleic acid), wherein the nucleic acid construct is operably associated with at least a control sequence (e.g., a promoter). Thus, some embodiments of the invention provide expression cassettes designed to express, for example, a nucleic acid construct of the invention. When an expression cassette comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). Thus, for example, a polynucleotide encoding an engineered protein, a polynucleotide encoding a deaminase (e.g., an adenine deaminase), and a polynucleotide comprising a guide nucleic acid comprised in an expression cassette may each be operably associated with a single promoter or one or more of the polynucleotide(s) may be operably associated with separate promoters (e.g., two or three promoters) in any combination, which may be the same or different from each other.

In some embodiments, an expression cassette comprising the polynucleotides/nucleic acid constructs of the invention may be optimized for expression in an organism (e.g., an animal, a plant, a bacterium and the like).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to a gene encoding a CRISPR-Cas effector protein or a gene encoding a deaminase, may be native to a host cell, or may be native to another source (e.g., foreign or heterologous to the promoter, to a gene encoding the CRISPR-Cas effector protein or a gene encoding the deaminase, to a host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

The expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). Thus, for example, a target nucleic acid may be contacted with a nucleic acid construct of the invention encoding, for example, a nucleic acid binding domain (e.g., a DNA binding domain such as a sequence-specific DNA binding protein (e.g., a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein)), a guide nucleic acid, and optionally a cytosine deaminase and/or adenine deaminase under conditions whereby the nucleic acid binding domain (e.g., a CRISPR-Cas effector protein) is expressed, and the nucleic acid binding domain forms a complex with the guide nucleic acid, the complex hybridizes to the target nucleic acid, and optionally the cytosine deaminase and/or adenine deaminase is/are recruited to the nucleic acid binding domain (and thus, to the target nucleic acid) or the cytosine deaminase and/or adenine deaminase are fused to the nucleic acid binding domain, thereby modifying the target nucleic acid. In some embodiments, the cytosine deaminase and/or adenine deaminase and the nucleic acid binding domain localize at the target nucleic acid, optionally through covalent and/or non-covalent interactions.

In some embodiments, a target nucleic acid may be contacted with a nucleic acid construct of the invention encoding an engineered protein, a guide nucleic acid, and optionally a cytosine deaminase and/or adenine deaminase under conditions whereby the engineered protein is expressed, or a target nucleic acid may be contacted with an engineered protein, a guide nucleic acid, and optionally a cytosine deaminase and/or adenine deaminase. The engineered protein can form a complex with the guide nucleic acid, and the complex can hybridize to the target nucleic acid, and optionally the cytosine deaminase and/or adenine deaminase is/are recruited to the engineered protein (and thus, to the target nucleic acid) or the cytosine deaminase and/or adenine deaminase are fused to the engineered protein, thereby modifying the target nucleic acid. The cytosine deaminase and/or adenine deaminase and the engineered protein may localize at the target nucleic acid, optionally through covalent and/or non-covalent interactions.

As used herein, “modifying” or “modification” in reference to a target nucleic acid includes editing (e.g., mutating), covalent modification, exchanging/substituting nucleic acids/nucleotide bases, deleting, cleaving, and/or nicking of a target nucleic acid to thereby provide a modified nucleic acid and/or altering transcriptional control of a target nucleic acid to thereby provide a modified nucleic acid. In some embodiments, a modification may include an insertion and/or deletion of any size and/or a single base change (SNP) of any type. In some embodiments, a modification comprises a SNP. In some embodiments, a modification comprises exchanging and/or substituting one or more (e.g., 1, 2, 3, 4, 5, or more) nucleotides. In some embodiments, an insertion or deletion may be about 1 base to about 30,000 bases in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000 bases in length or more, or any value or range therein). Thus, in some embodiments, an insertion or deletion may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 bases in length, or any range or value therein; about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 bases to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases or more in length, or any value or range therein; about 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases to about 2500, 3000, 3500, 4000, 4500, 5000, 5500, 6000, 6500, 7000, 7500, 8000, 8500, 9000, 9500, or 10,000 bases or more in length, or any value or range therein; or about 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 bases to about 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, or 5000 bases or more in length, or any value or range therein. In some embodiments, an insertion or deletion may be about 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, or 10,000 bases to about 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, or 30,000 bases or more in length, or any value or range therein.

“Recruit,” “recruiting” or “recruitment” as used herein refer to attracting one or more polypeptide(s) or polynucleotide(s) to another polypeptide or polynucleotide (e.g., to a particular location in a genome) using protein-protein interactions, nucleic acid protein interactions (e.g., RNA-protein interactions), and/or chemical interactions. Protein-protein interactions can include, but are not limited to, peptide tags (epitopes, multimerized epitopes) and corresponding affinity polypeptides, RNA recruiting motifs and corresponding affinity polypeptides, and/or chemical interactions. Example chemical interactions that may be useful with polypeptides and polynucleotides for the purpose of recruitment can include, but are not limited to, rapamycin-inducible dimerization of FRB-FKBP; Biotin-streptavidin interaction; SNAP tag (Hussain et al. Curr Pharm Des. 19(30):5437-42 (2013)); Halo tag (Los et al. ACS Chem Biol. 3(6):373-82 (2008)); CLIP tag (Gautier et al. Chemistry & Biology 15:128-136 (2008)); DmrA-DmrC heterodimer induced by a compound (Tak et al. Nat Methods 14(12):1163-1166 (2017)); Bifunctional ligand approaches (fuse two protein-binding chemicals together) (Voi et al. Curr Opin Chemical Biology 28:194-201 (2015)) (e.g. dihyrofolate reductase (DHFR) (Kopyteck et al. Cell Chem Biol 7(5):313-321 (2000)).

“Introducing,” “introduce,” “introduced” (and grammatical variations thereof) in the context of a polynucleotide of interest or editing system means presenting a nucleotide sequence of interest (e.g., polynucleotide, a nucleic acid construct, and/or a guide nucleic acid) and/or editing system (e.g., a polynucleotide, polypeptide, and/or ribonucleoprotein) to a host organism or cell of said organism (e.g., host cell; e.g., a plant cell) in such a manner that the nucleotide sequence and/or editing system gains access to the interior of a cell. Thus, for example, a nucleic acid construct of the invention encoding an engineered protein, a guide nucleic acid, and a cytosine deaminase and/or adenine deaminase may be introduced into a cell of an organism, thereby transforming the cell with the engineered protein, a guide nucleic acid, and a cytosine deaminase and/or adenine deaminase. In some embodiments, an engineered protein and/or a guide nucleic acid may be introduced into a cell of an organism, optionally wherein the engineered protein and guide nucleic acid may be comprised in a complex (e.g., a ribonucleoprotein). In some embodiments, the organism is a eukaryote (e.g., a mammal such as a human).

The term “transformation” as used herein refers to the introduction of a heterologous nucleic acid, polypeptide, and/or ribonucleoprotein into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct, a polypeptide, and/or a ribonucleoprotein of the invention.

“Transient transformation” in the context of a polynucleotide, polypeptide, and/or ribonucleoprotein means that a polynucleotide, polypeptide, and/or ribonucleoprotein is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromosomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention may be transiently introduced into a cell with a guide nucleic acid and as such, no DNA maintained in the cell.

A nucleic acid construct, polypeptide, and/or ribonucleoprotein of the invention can be introduced into a cell by any method known to those of skill in the art. In some embodiments, transformation methods include, but are not limited to, transformation via bacterial-mediated nucleic acid delivery (e.g., via Agrobacteria), viral-mediated nucleic acid delivery, silicon carbide and/or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the cell (e.g., a plant cell or an animal cell), including any combination thereof. In some embodiments of the invention, transformation of a cell comprises nuclear transformation. In some embodiments, transformation of a cell comprises plastid transformation (e.g., chloroplast transformation). In some embodiments, a recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

A nucleotide sequence, polypeptide, and/or ribonucleoprotein therefore can be introduced into a host organism or its cell in any number of ways that are well known in the art. The methods of the invention do not depend on a particular method for introducing one or more nucleotide sequence(s), polypeptide(s), and/or ribonucleoprotein(s) into the organism, only that they gain access to the interior of at least one cell of the organism. Where more than one nucleotide sequence, polypeptide, and/or ribonucleoprotein is to be introduced, they can be assembled as part of a single nucleic acid construct, or as separate nucleic acid constructs, and can be located on the same or different nucleic acid constructs. Accordingly, a nucleotide sequence, polypeptide, and/or ribonucleoprotein can be introduced into the cell of interest in a single transformation event, and/or in separate transformation events, or, alternatively, where relevant, a nucleotide sequence can be incorporated into a plant, for example, as part of a breeding protocol. In some embodiments, the cell is a eukaryotic cell (e.g., a mammalian such as a human cell or a plant cell).

In some embodiments, a nucleic acid construct of the invention (e.g., a polynucleotide encoding an engineered protein of the present invention, a polynucleotide encoding a deaminase, and/or a guide nucleic acid and/or expression cassettes and/or vectors comprising the same) may be operably linked to at least one regulatory sequence, optionally, wherein the at least one regulatory sequence may be codon optimized for expression in a plant. In some embodiments, the at least one regulatory sequence may be, for example, a promoter, an operon, a terminator, or an enhancer. In some embodiments, the at least one regulatory sequence may be a promoter. In some embodiments, the regulatory sequence may be an intron. In some embodiments, the at least one regulatory sequence may be, for example, a promoter operably associated with an intron or a promoter region comprising an intron. In some embodiments, the at least one regulatory sequence may be, for example a ubiquitin promoter and its associated intron (e.g., Medicago truncatula and/or Zea mays and their associated introns). In some embodiments, the at least one regulatory sequence may be a terminator nucleotide sequence and/or an enhancer nucleotide sequence.

In some embodiments, a nucleic acid construct of the invention may be operably associated with a promoter region, wherein the promoter region comprises an intron, optionally wherein the promoter region may be a ubiquitin promoter and intron (e.g., a Medicago or a maize ubiquitin promoter and intron, e.g., SEQ ID NO:48 or SEQ ID NO:49). In some embodiments, the nucleic acid construct of the invention that is operably associated with a promoter region comprising an intron may be codon optimized for expression in a plant.

In some embodiments, a nucleic acid construct of the invention may encode one or more (e.g., 1, 2, 3, 4, or more) polypeptide(s) of interest, optionally wherein the one or more polypeptides of interest may be codon optimized for expression in a plant. In some embodiments, an engineered protein may comprise to one or more (e.g., 1, 2, 3, 4, or more) polypeptide(s) of interest. For example, the heterologous polypeptide of an engineered protein may comprise or be a polypeptide of interest.

A polypeptide of interest useful with this invention can include, but is not limited to, a polypeptide or protein domain having deaminase activity, nickase activity, recombinase activity, transposase activity, methylase activity, glycosylase (DNA glycosylase) activity, glycosylase inhibitor activity (e.g., uracil-DNA glycosylase inhibitor (UGI)), demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, a nuclear localization sequence or activity, an affinity polypeptide, a peptide tag, and/or photolyase activity. In some embodiments, the polypeptide of interest is a Fok1 nuclease, or a uracil-DNA glycosylase inhibitor. When encoded in a nucleic acid (polynucleotide, expression cassette, and/or vector) the encoded polypeptide or protein domain may be codon optimized for expression in an organism. In some embodiments, a polypeptide of interest may be linked to an engineered protein of the present invention or CRISPR-Cas effector protein domain to provide a CRISPR-Cas fusion protein. In some embodiments, a CRISPR-Cas fusion protein that comprises a CRISPR-Cas effector protein domain linked to a peptide tag may also be linked to a polypeptide of interest (e.g., a CRISPR-Cas effector protein domain may be, for example, linked to both a peptide tag (or an affinity polypeptide) and, for example, a polypeptide of interest.

In some embodiments, an editing system of the present invention comprises a CRISPR-Cas effector protein. As used herein, a “CRISPR-Cas effector protein” is a protein or polypeptide that cleaves, cuts, or nicks a nucleic acid; binds a nucleic acid (e.g., a target nucleic acid and/or a guide nucleic acid); and/or that identifies, recognizes, or binds a guide nucleic acid as defined herein. In some embodiments, a CRISPR-Cas effector protein may be an enzyme (e.g., a nuclease, endonuclease, nickase, etc.) and/or may function as an enzyme. In some embodiments, a CRISPR-Cas effector protein refers to a CRISPR-Cas nuclease. In some embodiments, a CRISPR-Cas effector protein comprises nuclease activity and/or nickase activity, comprises a nuclease domain whose nuclease activity and/or nickase activity has been reduced or eliminated, comprises single stranded DNA cleavage activity (ss DNAse activity) or which has ss DNAse activity that has been reduced or eliminated, and/or comprises self-processing RNAse activity or which has self-processing RNAse activity that has been reduced or eliminated. A CRISPR-Cas effector protein may bind to a target nucleic acid. A CRISPR-Cas effector protein may be a Type I, II, III, IV, V, or VI CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein. In some embodiments, a CRISPR-Cas effector protein may be Cas12a and optionally may have an amino acid sequence of any one of SEQ ID NOs:50-66 and/or a nucleotide sequence of any one of SEQ ID NOs:67-69. In some embodiments, a CRISPR-Cas effector protein may be an active Cas12a and optionally may have an amino acid sequence of SEQ ID NO:58. In some embodiments, a CRISPR-Cas effector protein may be an inactive (i.e., dead) Cas12a and optionally may have an amino acid sequence of SEQ ID NO:50. In some embodiments, a CRISPR-Cas effector protein may be Cas12b and optionally may have an amino acid sequence of SEQ ID NO:151.

Exemplary CRISPR-Cas effector proteins include, but are not limited to, a Cas9, C2c1, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site and/or nuclease domain (e.g., RuvC, HNH, e.g., a RuvC site of a Cas12a nuclease domain; e.g., a RuvC site and/or HNH site of a Cas9 nuclease domain). A CRISPR-Cas effector protein having a mutation in its nuclease active site and/or nuclease domain, and therefore, no longer comprising nuclease activity, is commonly referred to as “inactive” or “dead,” e.g., dCas9. In some embodiments, a CRISPR-Cas effector protein having a mutation in its nuclease active site and/or nuclease domain may have impaired activity or reduced activity (e.g., nickase activity) as compared to the same CRISPR-Cas effector protein without the mutation.

A CRISPR Cas9 effector protein or Cas9 useful with this invention may be any known or later identified Cas9 nuclease. In some embodiments, a Cas9 of the present invention may be a protein from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. In some embodiments, a CRISPR-Cas effector protein may be a Cas9 and optionally may have a nucleotide sequence of any one of SEQ ID NOs:70-80 or 140-143 and/or an amino acid sequence of any one of SEQ ID NOs:81-82.

In some embodiments, the CRISPR-Cas effector protein may be a Cas9 derived from Streptococcus pyogenes and/or may recognize the PAM sequence motif NGG, NAG, NGA (Mali et al, Science 2013; 339(6121): 823-826). In some embodiments, the CRISPR-Cas effector protein may be a Cas9 derived from Streptococcus thermophiles and/or may recognize the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 derived from Streptococcus mutans and/or may recognize the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 derived from Streptococcus aureus and/or may recognize the PAM sequence motif NNGRR (R=A or G). In some embodiments, the CRISPR-Cas effector protein may be a Cas9 derived from S. aureus and/or may recognize the PAM sequence motif N GRRT (R=A or G). In some embodiments, the CRISPR-Cas effector protein may be a Cas9 derived from S. aureus and/or may recognize the PAM sequence motif N GRRV (R=A or G). In some embodiments, the CRISPR-Cas effector protein may be a Cas9 that is derived from Neisseria meningitidis and/or may recognize the PAM sequence motif N GATT or N GCTT (R=A or G, V=A, G or C) (See, e.g., Hou et ah, PNAS 2013, 1-6). In the aforementioned embodiments in this paragraph, N in the PAM sequence motif can be any nucleotide residue, e.g., any of A, G, C or T. In some embodiments, the CRISPR-Cas effector protein may be a Cas13a derived from Leptotrichia shahii and/or may recognize a protospacer flanking sequence (PFS) (or RNA PAM (rPAM)) sequence motif of a single 3′ A, U, or C, which may be located within the target nucleic acid.

A Type V CRISPR-Cas effector protein useful with embodiments of the invention may be any Type V CRISPR-Cas nuclease. Exemplary Type V CRISPR-Cas effector proteins include, but are not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas effector protein may be a Cas12a. In some embodiments, a Type V CRISPR-Cas effector protein may be a nickase, optionally, a Cas12a nickase. In some embodiments, a Type V CRISPR-Cas effector protein may be a Cas12b (e.g., SEQ ID NO:151).

In some embodiments, the CRISPR-Cas effector protein may be a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein useful with this invention may be any known or later identified Cas12a (previously known as Cpf1) (see, e.g., U.S. Pat. No. 9,790,490, which is incorporated by reference for its disclosures of Cpf1 (Cas12a) sequences). The term “Cas12a” refers to an RNA-guided protein that can have nuclease activity, the protein comprising a guide nucleic acid binding domain and an active, inactive, or partially active DNA cleavage domain, thereby the RNA-guided nuclease activity of the Cas12a may be active, inactive or partially active, respectively. In some embodiments, a Cas12a useful with the invention may comprise a mutation in the nuclease active site (e.g., RuvC site of the Cas12a domain). A Cas12a having a mutation in its nuclease domain and/or nuclease active site, and therefore, no longer comprising nuclease activity, is commonly referred to as deadCas12a (e.g., dCas12a). In some embodiments, a Cas12a having a mutation in its nuclease domain and/or nuclease active site may have impaired activity, e.g., may have reduced nickase activity.

In some embodiments, a CRISPR-Cas effector protein may be optimized for expression in an organism, for example, in an animal (e.g., a mammal such as a human), a plant, a fungus, an archaeon, or a bacterium. In some embodiments, a CRISPR-Cas effector protein (e.g., Cas12a polypeptide/domain or a Cas9 polypeptide/domain) may be optimized for expression in a plant.

Any deaminase domain/polypeptide useful for base editing may be used with this invention. A “cytosine deaminase” and “cytidine deaminase” as used herein refer to a polypeptide or domain thereof that catalyzes or is capable of catalyzing cytosine deamination in that the polypeptide or domain catalyzes or is capable of catalyzing the removal of an amine group from a cytosine base. Thus, a cytosine deaminase may result in conversion of cytosine to a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid. In some embodiments, a cytosine deaminase encoded by a polynucleotide of the invention generates a C to T, G, or A conversion in the complementary strand in the genome.

A cytosine deaminase useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally-occurring cytosine deaminase, including, but not limited to, a primate (e.g., a human, monkey, chimpanzee, gorilla), a dog, a cow, a rat or a mouse. Thus, in some embodiments, an cytosine deaminase useful with the invention may be about 70% to about 100% identical to a wild-type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDAI (e.g., At2g19570), and evolved versions of the same. Evolved deaminases are disclosed in, for example, U.S. Pat. No. 10,113,163, Gaudelli et al. Nature 551(7681):464-471 (2017)) and Thuronyi et al. (Nature Biotechnology 37: 1070-1079 (2019)), each of which are incorporated by reference herein for their disclosure of deaminases and evolved deaminases. In some embodiments, the cytosine deaminase may be an APOBEC1 deaminase having the amino acid sequence of SEQ ID NO:83. In some embodiments, the cytosine deaminase may be an APOBEC3A deaminase having the amino acid sequence of SEQ ID NO:84. In some embodiments, the cytosine deaminase may be an CDA1 deaminase, optionally a CDA1 having the amino acid sequence of SEQ ID NO:85. In some embodiments, the cytosine deaminase may be a FERNY deaminase, optionally a FERNY having the amino acid sequence of SEQ ID NO:86. In some embodiments, the cytosine deaminase may be a rAPOBEC1 deaminase, optionally a rAPOBEC1 deaminase having the amino acid sequence of SEQ ID NO:87. In some embodiments, the cytosine deaminase may be a hAID deaminase, optionally a hAID having the amino acid sequence of SEQ ID NO:88 or SEQ ID NO:89. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., “evolved deaminases”) (see, e.g., SEQ ID NO:90, SEQ ID NO:91, SEQ ID NO:92). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of any one of SEQ ID NOs:83-92 (e.g., at least 80%, at least 85%, at least 90%, at least 92%, at least 95%, at least 96%, at least 97%, at least 98%, at least 99%, or at least 99.5% identical to the amino acid sequence of any one of SEQ ID NOs:83-92). In some embodiments, a polynucleotide encoding a cytosine deaminase may be codon optimized for expression in a plant and the codon optimized polypeptide may be about 70% to 99.5% identical to the reference polynucleotide.

An “adenine deaminase” and “adenosine deaminase” as used herein refer to a polypeptide or domain thereof that catalyzes or is capable of catalyzing the hydrolytic deamination (e.g., removal of an amine group from adenine) of adenine or adenosine. In some embodiments, an adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A-G conversion in the sense (e.g., “+”; template) strand of the target nucleic acid or a T-C conversion in the antisense (e.g., “−”, complementary) strand of the target nucleic acid. An adenine deaminase useful with this invention may be any known or later identified adenine deaminase from any organism (see, e.g., U.S. Pat. No. 10,113,163, which is incorporated by reference herein for its disclosure of adenine deaminases).

In some embodiments, an adenosine deaminase may be a variant of a naturally-occurring adenine deaminase. Thus, in some embodiments, an adenosine deaminase may be about 70% to 100% identical to a wild-type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild-type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild-type E. coli TadA comprises the amino acid sequence of SEQ ID NO:93. In some embodiments, a mutated/evolved E. coli TadA* comprises the amino acid sequence of any one of SEQ ID NOs:94-97. In some embodiments, a polynucleotide encoding a TadA/TadA* may be codon optimized for expression in a plant. In some embodiments, an adenine deaminase may comprise all or a portion of an amino acid sequence of any one of SEQ ID NOs:98-103. In some embodiments, an adenine deaminase may comprise all or a portion of an amino acid sequence of any one of SEQ ID NOs:93-103.

In some embodiments, a nucleic acid construct of this invention may further encode a glycosylase inhibitor (e.g., a uracil glycosylase inhibitor (UGI) such as uracil-DNA glycosylase inhibitor). In some embodiments, the invention provides fusion proteins comprising an engineered protein and a UGI and/or one or more polynucleotides encoding the same, optionally wherein the one or more polynucleotides may be codon optimized for expression in a plant.

A “uracil glycosylase inhibitor” useful with the invention may be any protein or polypeptide that is capable of inhibiting a uracil-DNA glycosylase base-excision repair enzyme. In some embodiments, a UGI domain comprises a wild-type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:104 or a polypeptide having about 70% to about 99.5% identity to the amino acid sequence of SEQ ID NO:104 (e.g., at least 80%, at least 85%, at least 90%, at least 92%, at least 95%, at least 96%, at least 97%, at least 98%, at least 99%, or at least 99.5% identical to the amino acid sequence of SEQ ID NO:104). For example, in some embodiments, a UGI domain may comprise a fragment of the amino acid sequence of SEQ ID NO:104 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:104. In some embodiments, a UGI domain may be a variant of a known UGI (e.g., SEQ ID NO:104) having about 70% to about 99.5% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An engineered protein may be used in combination with a guide nucleic acid (e.g., guide RNA (gRNA), CRISPR array, CRISPR RNA, crRNA) that is designed to function with the engineered protein to modify a target nucleic acid. A guide nucleic acid useful with this invention may comprise at least one spacer sequence and at least one repeat sequence. The guide nucleic acid is capable of forming a complex with the engineered protein (e.g., with a nuclease domain of the engineered protein) and the spacer sequence is capable of hybridizing to a target nucleic acid, thereby guiding the complex to the target nucleic acid, wherein the target nucleic acid may be modified (e.g., cleaved or edited) and/or modulated (e.g., modulating transcription) by a deaminase (e.g., a cytosine deaminase and/or adenine deaminase, optionally present in and/or recruited to the complex).

In some embodiments, an engineered protein comprising a Cas9 domain (or a nucleic acid construct encoding the same) may be used in combination with a Cas9 guide nucleic acid to modify a target nucleic acid, and a deaminase (e.g., cytosine and/or adenine) may be linked to or form a complex with the engineered protein. A cytosine deaminase deaminates a cytosine base in the target nucleic acid, thereby editing the target nucleic acid. An adenine deaminase deaminates an adenosine base in the target nucleic acid, thereby editing the target nucleic acid.

Likewise, an engineered protein may comprise a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csxl2), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, Csf4 (dinG), and/or Csf5), which may form a complex with or be linked to a cytosine deaminase domain and/or adenine deaminase domain, and may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base or adenosine base, respectively, in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof, a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csxl2), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, Csf4 (dinG), and/or Csf5, or a fragment thereof), wherein the repeat sequence may be linked to the 5′ end and/or the 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35 (Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild-type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild-type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be fully complementary to a target nucleic acid, while the 3′ region of the spacer may be substantially complementary to the target nucleic acid (such as for a spacer in a Type V CRISPR-Cas system), or the 3′ region of a spacer sequence of a guide nucleic acid may be fully complementary to a target nucleic acid, while the 5′ region of the spacer may be substantially complementary to the target nucleic acid (such as for a spacer in a Type II CRISPR-Cas system), and therefore, the overall complementarity of the spacer sequence to the target nucleic acid may be less than 100%. Thus, for example, in a guide nucleic acid for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target nucleic acid, while the remaining nucleotides in the 3′ region of the spacer sequence are substantially complementary (e.g., at least about 70% complementary) to the target nucleic acid. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target nucleic acid, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleic acid.

As a further example, in a guide nucleic acid for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target nucleic acid, while the remaining nucleotides in the 5′ region of the spacer sequence are substantially complementary (e.g., at least about 70% complementary) to the target nucleic acid. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target nucleic acid, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target nucleic acid. A recruiting guide RNA further comprises one or more recruiting motifs as described herein, which may be linked to the 5′ end of the guide or the 3′ end or it may be inserted into the recruiting guide nucleic acid (e.g., within the hairpin loop).

In some embodiments, a seed region of a spacer may be about 8 to about 10 nucleotides in length, about 5 to about 6 nucleotides in length, or about 6 nucleotides in length.

A “target nucleic acid”, “target DNA,” “target nucleotide sequence,” “target region,” and “target region in the genome” are used interchangeably herein and refer to a region of an organism's (e.g., a plant's) genome that comprises a sequence that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid as defined herein. A target nucleic acid is targeted by an editing system (or a component thereof) as described herein. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome or mammalian (e.g., human) genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” or “protospacer” as used herein refer to a sequence that is fully or substantially complementary to (and can hybridize to) a spacer sequence of a guide nucleic acid. In some embodiments, the protospacer is all or a portion of a target nucleic acid as defined herein that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

5′-NNNNNNNNNNNNNNNNNNN-3′ RNA Spacer (SEQ ID NO:105)
   | | | | | | | | | | | | | | | | | | | |

3′AAANNNNNNNNNNNNNNNNNNN-5′ Target strand (SEQ ID NO:106)
   | | | |

5′TTTNNNNNNNNNNNNNNNNNNN-3′ Non-target strand (SEQ ID NO:107)

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encodes an engineered protein, and/or a deaminase, and each may be comprised on the same or on a separate expression cassette or vector from that comprising the one or more guide nucleic acids. When the nucleic acid construct encoding an engineered protein or the components of an editing system is/are comprised on separate expression cassette(s) or vector(s) from that comprising the guide nucleic acid, a target nucleic acid may be contacted with (e.g., provided with) the expression cassette(s) or vector(s) encoding the engineered protein or components of an editing system in any order from one another and the guide nucleic acid, e.g., prior to, concurrently with, or after the expression cassette comprising the guide nucleic acid is provided (e.g., contacted with the target nucleic acid).

Methods of recruiting one or more components of an editing system to each other and/or to a target nucleic acid are known in the art and may include the use of a peptide tag or an affinity polypeptide that interacts with the peptide tag. In some embodiments, a guide nucleic acid may be linked to an RNA recruiting motif and a deaminase may be linked to an affinity polypeptide capable of interacting with the RNA recruiting motif, thereby recruiting the deaminase to the target nucleic acid. Alternatively, chemical interactions may be used to recruit a polypeptide (e.g., a deaminase) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases).

In some embodiments of the invention, a guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Con RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF). Exemplary RNA recruiting motifs and corresponding affinity polypeptides that may be useful with this invention can include, but are not limited to, SEQ ID NOs:108-118.

In some embodiments, the components for recruiting polypeptides and nucleic acids may include those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together; e.g. dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

As described herein, a “peptide tag” may be employed to recruit one or more polypeptides. A peptide tag may be any polypeptide that is capable of being bound by a corresponding affinity polypeptide. A peptide tag may also be referred to as an “epitope” and when provided in multiple copies, a “multimerized epitope.” Example peptide tags can include, but are not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO2018/136783 and U.S. Patent Application Publication No. 2017/0219596, which are incorporated by reference for their disclosures of peptide tags. Peptide tags that may be useful with this invention can include, but are not limited to, SEQ ID NO:119 and SEQ ID NO:120. An affinity polypeptide useful with peptide tags includes, but is not limited to, SEQ ID NO:121.

A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20 or more amino acids, optionally about 3 to about 10, about 4 to about 10, about 5 to about 10, about 5 to about 15, or about 5 to about 20 amino acids, and the like, and any value or range therein. Thus, in some embodiments, a CRISPR-Cas effector protein of the invention may comprise a CRISPR-Cas effector protein domain fused to one peptide tag or to two or more peptide tags, optionally wherein the two or more peptide tags are fused to one another via one or more amino acid residues. In some embodiments, a peptide tag useful with the invention may be a single copy of a GCN4 peptide tag or epitope or may be a multimerized GCN4 epitope comprising about 2 to about 25 or more copies of the peptide tag (e.g., about 2, 3, 4, 5, 6, 7, 8, 9, 10, 11, 12, 13, 14, 15, 16, 17, 18, 19, 20, 21, 22, 23, 24, 25 or more copies of a GCN4 epitope or any range therein).

In some embodiments, a peptide tag may be fused to a CRISPR-Cas polypeptide or domain. In some embodiments, a peptide tag may be fused or linked to the C-terminus of a CRISPR-Cas effector protein to form a CRISPR-Cas fusion protein. In some embodiments, a peptide tag may be fused or linked to the N-terminus of a CRISPR-Cas effector protein to form a CRISPR-Cas fusion protein. In some embodiments, a peptide tag may be fused within a CRISPR-Cas effector protein (e.g., a peptide tag may be in a loop region of a CRISPR-Cas effector protein). In some embodiments, peptide tag may be fused to a cytosine deaminase and/or to an adenine deaminase.

An “affinity polypeptide” (e.g., “recruiting polypeptide”) refers to any polypeptide that is capable of binding to its corresponding peptide tag, peptide tag, or RNA recruiting motif. An affinity polypeptide for a peptide tag may be, for example, an antibody and/or a single chain antibody that specifically binds the peptide tag, respectively. In some embodiments, an antibody for a peptide tag may be, but is not limited to, an scFv antibody. In some embodiments, an affinity polypeptide may be fused or linked to the N-terminus of a deaminase (e.g., a cytosine deaminase or an adenine deaminase). In some embodiments, the affinity polypeptide is stable under the reducing conditions of a cell or cellular extract.

The nucleic acid constructs of the invention and/or guide nucleic acids may be comprised in one or more expression cassettes as described herein. In some embodiments, a nucleic acid construct of the invention may be comprised in the same or in a separate expression cassette or vector from that comprising a guide nucleic acid and/or a recruiting guide nucleic acid.

When used in combination with guide nucleic acids and recruiting guide nucleic acids, the nucleic acid constructs of the invention (and expression cassettes and vectors comprising the same) may be used to modify a target nucleic acid and/or its expression. A target nucleic acid may be contacted with a nucleic acid construct of the invention and/or expression cassettes and/or vectors comprising the same prior to, concurrently with or after contacting the target nucleic acid with the guide nucleic acid/recruiting guide nucleic acid (and/or expression cassettes and vectors comprising the same.

According to embodiments of the present invention, provided are engineered proteins. An “engineered protein” as used herein refers to a polypeptide that comprises a polypeptide from a CRISPR-Cas effector protein (i.e., a CRISPR-Cas effector polypeptide) and a polypeptide that is heterologous to the CRISPR-Cas effector polypeptide (i.e., a heterologous polypeptide). The polypeptide from a CRISPR-Cas effector protein is referred to herein as a “CRISPR-Cas effector polypeptide” and a “CRISPR-Cas effector polypeptide” is a portion of a CRISPR-Cas effector protein. Accordingly, a “CRISPR-Cas effector polypeptide” as used herein does not include all of a CRISPR-Cas effector protein and, thus, has a reduced number of amino acids compared to the number of amino acids for the CRISPR-Cas effector protein. In some embodiments, a CRISPR-Cas effector polypeptide is devoid of a nuclease domain (e.g., devoid of a RuvC domain). The polypeptide that is heterologous to the CRISPR-Cas effector polypeptide is referred to herein as a heterologous polypeptide. The heterologous polypeptide may be a polypeptide of interest as described herein. In some embodiments, an engineered protein comprises all or a portion of a deaminase domain (e.g., a cytosine deaminase and/or adenine deaminase), which may be linked to any portion of the engineered protein. For example, in some embodiments, all or a portion of a deaminase domain is linked to the N- or C-terminus of the CRISPR-Cas effector polypeptide and/or to the N- or C-terminus of the engineered protein. In some embodiments, all or a portion of a deaminase domain is between two portions of an engineered protein. An engineered protein can cleave, cut, or nick a nucleic acid; bind a nucleic acid (e.g., a target nucleic acid and/or a guide nucleic acid); and/or identify, recognize, or bind a guide nucleic acid as defined herein. In some embodiments, an engineered protein or a portion thereof may be an enzyme (e.g., a nuclease, endonuclease, nickase, etc.) and/or may function as an enzyme. In some embodiments, an engineered protein of the present invention is an RNA-guided DNA-binding protein. In some embodiments, an engineered protein is present in and/or forms a complex with a guide nucleic acid that is a single guide nucleic acid (e.g., a gRNA, CRISPR array, and/or crRNA), optionally wherein the guide nucleic acid is a single crRNA. In some embodiments, a complex comprises an engineered protein and a guide nucleic acid and the guide nucleic acid and/or complex consists of a single guide nucleic acid (e.g., a single crRNA). In some embodiments, an engineered protein binds a single guide nucleic acid (e.g., a single crRNA), recognizes and/or binds a target nucleic acid, and has nuclease activity, optionally wherein the engineered protein cleaves the target strand of the target nucleic acid.

In some embodiments, an engineered protein comprises a first CRISPR-Cas effector polypeptide and a heterologous polypeptide. The first CRISPR-Cas effector polypeptide may be devoid of a nuclease domain, optionally devoid of a RuvC domain. The heterologous polypeptide may be linked to the N- or C-terminus of the first CRISPR-Cas effector polypeptide, optionally with or without a linker (e.g., a peptide linker). In some embodiments, the first CRISPR-Cas effector polypeptide is a portion of a first CRISPR-Cas effector protein (e.g., a portion of a Type V CRISPR-Cas effector protein such as a portion of a Cas12a). In some embodiments, the heterologous polypeptide comprises a nuclease domain, optionally a HNH domain (e.g., an HNH domain comprising a sequence having at least 70%, 75%, 80%, 85%, 90%, 95%, 96%, 97%, 98%, 99%, or 100% sequence identity to the amino acid sequence of one or more of SEQ ID NOs:1 or 169-174). In some embodiments, the heterologous polypeptide comprises a HNH domain that is from a CRISPR-Cas effector protein and/or comprises a sequence having at least 70%, 75%, 80%, 85%, 90%, 95%, 96%, 97%, 98%, 99%, or 100% sequence identity to the amino acid sequence of one of SEQ ID NOs:1 or 172. In some embodiments, the heterologous polypeptide comprises a HNH domain that is not from a CRISPR-Cas effector protein and/or comprises a sequence having at least 70%, 75%, 80%, 85%, 90%, 95%, 96%, 97%, 98%, 99%, or 100% sequence identity to the amino acid sequence of one of SEQ ID NOs:169-171 or 173-174. In some embodiments, the heterologous polypeptide is a polypeptide from a CRISPR-Cas effector protein, optionally wherein the heterologous polypeptide is from a different type of CRISPR-Cas effector protein (e.g., a Type II CRISPR-Cas effector protein) than the type of the first CRISPR-Cas effector protein (e.g., a Type IV CRISPR-Cas effector protein) from which the first CRISPR-Cas effector polypeptide is a portion of.

In some embodiments, an engineered protein comprises a first CRISPR-Cas effector polypeptide, a heterologous polypeptide, and a second CRISPR-Cas effector polypeptide, which may be linked together in any order. In some embodiments, the first CRISPR-Cas effector polypeptide may be devoid of a RuvC domain. The heterologous polypeptide may be linked to the N- or C-terminus of the first CRISPR-Cas effector polypeptide, optionally with or without a linker (e.g., a peptide linker), and/or the heterologous polypeptide may be linked to the N- or C-terminus of the second CRISPR-Cas effector polypeptide, optionally with or without a linker (e.g., a peptide linker). In some embodiments, the heterologous polypeptide is between the first CRISPR-Cas effector polypeptide and the second CRISPR-Cas effector polypeptide. In some embodiments, the first CRISPR-Cas effector polypeptide is a portion of a first CRISPR-Cas effector protein (e.g., a portion of a Type V CRISPR-Cas effector protein such as a portion of a Cas12a) and the second CRISPR-Cas effector polypeptide is a portion of a second CRISPR-Cas effector protein (e.g., a portion of a Type V CRISPR-Cas effector protein such as a portion of a Cas12a), wherein the first CRISPR-Cas effector protein and second CRISPR-Cas effector protein may be the same protein or different proteins. In some embodiments, the first CRISPR-Cas effector protein and the second CRISPR-Cas effector protein are the same, thereby the first CRISPR-Cas effector polypeptide and second CRISPR-Cas effector polypeptide are portions from the same protein, but may be different portions of the CRISPR-Cas effector protein. The first CRISPR-Cas effector polypeptide and second CRISPR-Cas effector polypeptide may have different sequences. In some embodiments, first CRISPR-Cas effector polypeptide and second CRISPR-Cas effector polypeptide may comprise a sequence that is the same. In some embodiments, the first CRISPR-Cas effector polypeptide and second CRISPR-Cas effector polypeptide together provide the full sequence of the CRISPR-Cas effector protein. In some embodiments, the first CRISPR-Cas effector polypeptide and second CRISPR-Cas effector polypeptide together do not make up the full sequence of the CRISPR-Cas effector protein (i.e., a portion of the sequence of the CRISPR-Cas effector protein is not present in the two sequences of the first CRISPR-Cas effector polypeptide and second CRISPR-Cas effector polypeptide); for example, 1 or 5 to 10, 15, 20, 25, 30, or more amino acids (e.g., 1, 2, 3, 4, 5, 6, 7, 8, 9, 10, 15, 20, 25, 30, or more amino acid(s)) of the CRISPR-Cas effector protein may not be present in the sequences of the first and second CRISPR-Cas effector polypeptides. In some embodiments, the heterologous polypeptide comprises a nuclease domain, optionally a HNH domain (e.g., an HNH domain from a Type II CRISPR-Cas effector protein). In some embodiments, the heterologous polypeptide comprises a HNH domain that is not from a CRISPR-Cas effector protein. In some embodiments, the heterologous polypeptide is a polypeptide from a CRISPR-Cas effector protein, optionally wherein the heterologous polypeptide is from a different type of CRISPR-Cas effector protein (e.g., a Type II CRISPR-Cas effector protein) than the type of the first CRISPR-Cas effector protein (e.g., a Type IV CRISPR-Cas effector protein) from which the first CRISPR-Cas effector polypeptide is a portion of and/or than the type of the second CRISPR-Cas effector protein (e.g., a Type IV CRISPR-Cas effector protein) from which the second CRISPR-Cas effector polypeptide is a portion of. In some embodiments, the heterologous polypeptide is from a Type II CRISPR-Cas effector protein (e.g., is a portion (e.g., the HNH domain or a portion thereof) of the Type II CRISPR-Cas effector protein), the first CRISPR-Cas effector polypeptide is a portion of a Type IV CRISPR-Cas effector protein, and the second CRISPR-Cas effector polypeptide is a portion of a Type IV CRISPR-Cas effector protein, wherein the first and second CRISPR-Cas effector polypeptides are different. In some embodiments, the heterologous polypeptide is heterologous to one of the first CRISPR-Cas effector polypeptide and second CRISPR-Cas effector polypeptide. In some embodiments, the heterologous polypeptide is heterologous to both the first CRISPR-Cas effector polypeptide and the second CRISPR-Cas effector polypeptide.

“Heterologous polypeptide” as used herein refers to a non-naturally occurring polypeptide compared to a CRISPR-Cas effector polypeptide of an engineered protein. Accordingly, a heterologous polypeptide of an engineered protein is not found in nature in at least one CRISPR-Cas effector polypeptide of the engineered protein, so the heterologous polypeptide is non-naturally occurring with respect the at least one CRISPR-Cas effector polypeptide. For example, an engineered protein of the present invention includes a CRISPR-Cas effector polypeptide that is a portion of a CRISPR-Cas effector protein and the engineered protein includes a heterologous polypeptide, and the heterologous polypeptide is non-naturally occurring compared to the CRISPR-Cas effector polypeptide in the absence of the heterologous polypeptide (e.g., the CRISPR-Cas effector polypeptide without or prior to including (e.g., insertion or fusion of) the heterologous polypeptide and the CRISPR-Cas effector polypeptide); in some embodiments, the heterologous polypeptide is heterologous to the CRISPR-Cas effector protein from which the CRISPR-Cas effector polypeptide is a portion of. In some embodiments, an engineered protein includes a heterologous polypeptide, a first CRISPR-Cas effector polypeptide that is a portion of a first CRISPR-Cas effector protein, and a second CRISPR-Cas effector polypeptide that is a portion of a second CRISPR-Cas effector protein, and the heterologous polypeptide is non-naturally occurring in (i.e., heterologous to) the first CRISPR-Cas effector polypeptide, the first CRISPR-Cas effector protein, the second CRISPR-Cas effector, and the second CRISPR-Cas effector protein. Similarly, a nucleotide sequence encoding a heterologous polypeptide is heterologous to (i.e., non-naturally occurring compared to) a nucleotide sequence encoding a CRISPR-Cas effector polypeptide of an engineered protein.

In some embodiments, the heterologous polypeptide comprises a polypeptide or domain from a different type of protein than a CRISPR-Cas effector polypeptide of the engineered protein. In some embodiments, an engineered protein comprises one or more (e.g., 1, 2, 3, or more) portion(s) of (i.e., one or more CRISPR-Cas effector polypeptide(s) from) a Type V CRISPR-Cas effector protein (e.g., Cas 12a) and one or more (e.g., 1, 2, 3, or more) polypeptide(s) from a different type of CRISPR-Cas effector protein such as a Type II CRISPR-Cas effector protein. When two or more portions or polypeptides are from the same protein and each are present in an engineered protein, the two or more portions or polypeptides may be separated from each other in the engineered protein by a linker and/or a heterologous polypeptide (i.e., the two or more portions or polypeptides may not be directly linked) or may be in a different order than that of the protein from which they are from (e.g., a wild-type protein and/or CRISPR-Cas effector protein). In some embodiments, an engineered protein comprises one or more (e.g., 1, 2, 3, or more) portion(s) of (i.e., one or more CRISPR-Cas effector polypeptide(s) from) a Type V CRISPR-Cas effector protein (e.g., Cas 12a) and at least one polypeptide from and/or portion of a Type II CRISPR-Cas effector protein (e.g., Cas 9). In some embodiments, an engineered protein comprises a first CRISPR-Cas effector polypeptide that is a portion of a Type V CRISPR-Cas effector protein (e.g., Cas 12a) and a heterologous polypeptide from a Type II CRISPR-Cas effector protein (e.g., Cas 9). In some embodiments, an engineered protein comprises a first CRISPR-Cas effector polypeptide that is a portion of a Type V CRISPR-Cas effector protein (e.g., Cas 12a), a heterologous polypeptide from a Type II CRISPR-Cas effector protein (e.g., Cas 9), and a second CRISPR-Cas effector polypeptide that is a portion of a Type V CRISPR-Cas effector protein (e.g., Cas 12a), optionally wherein the first and second CRISPR-Cas effector polypeptides are different portions from the same Type V CRISPR-Cas effector protein (e.g., Cas 12a). In some embodiments, an engineered protein comprises a first CRISPR-Cas effector polypeptide that is a portion of a Type V CRISPR-Cas effector protein (e.g., Cas 12a), a heterologous polypeptide that comprises a HNH domain or a portion thereof, and a second CRISPR-Cas effector polypeptide that is a portion of a Type V CRISPR-Cas effector protein (e.g., Cas 12a), optionally wherein the first and second CRISPR-Cas effector polypeptides are different portions from the same Type V CRISPR-Cas effector protein (e.g., Cas 12a).

In some embodiments, an engineered protein comprises one or more (e.g., 1, 2, 3, or more) domain(s) or a portion thereof from a Type V CRISPR-Cas effector protein (e.g., Cas 12a) and one or more (e.g., 1, 2, 3, or more) domain(s) or a portion thereof from a different type of CRISPR-Cas effector protein such as a Type II CRISPR-Cas effector protein. In some embodiments, an engineered protein comprises one or more (e.g., 1, 2, 3, or more) domain(s) or a portion thereof from a Type V CRISPR-Cas effector protein (e.g., Cas 12a) and at least one domain or a portion thereof from a Type II CRISPR-Cas effector protein (e.g., Cas 9). In some embodiments, the heterologous polypeptide of an engineered protein does not interfere or adversely affect the activity of a CRISPR-Cas effector polypeptide and/or of one or more domain(s) of the CRISPR-Cas effector polypeptide (e.g., a RuvC domain).

The heterologous polypeptide may have a length of about 10 to about 300 amino acids such as about 10, 20, 30, 40, 50, 60, 70, 80, 90, or 100 amino acids to about 110, 125, 150, 175, 200, 225, 250, 275, or 300 amino acids. In some embodiments, the heterologous polypeptide has a length of about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 amino acids. In some embodiments, the heterologous polypeptide has a length of about 120, 125, 130, 135, or 140 amino acids to about 145, 150, 155, or 160 amino acids. In some embodiments, the heterologous polypeptide has a length of 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, or 160 amino acids. In some embodiments, the heterologous polypeptide is between a first CRISPR-Cas effector polypeptide and a second CRISPR-Cas effector polypeptide and the heterologous polypeptide is heterologous to one or both of the first and second CRISPR-Cas effector polypeptides.

In some embodiments, a CRISPR-Cas effector polypeptide has a length of about 100, 150, 200, or 250 amino acids to about 300, 350, or 400 amino acids. In some embodiments, a CRISPR-Cas effector polypeptide has a length of about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 amino acids. In some embodiments, a CRISPR-Cas effector polypeptide has a length of about 800, 850, or 900 amino acids to about 950, 1,000, 1,050, or 1,100 amino acids. In some embodiments, a CRISPR-Cas effector polypeptide has a length of about 800, 810, 820, 830, 840, 850, 860, 870, 880, 890, 900, 910, 920, 930, 940, 950, 960, 970, 980, 990, 1,000, 1,010, 1,020, 1,030, 1,040, 1,050, 1,060, 1,070, 1,080, 1,090, 1,100 amino acids. In some embodiments, an engineered protein comprises a first CRISPR-Cas effector polypeptide having a length of about 100, 150, 200, or 250 amino acids to about 300, 350, or 400 amino acids, a heterologous polypeptide having a length of about 10, 50, 100, or 140 amino acids to about 160, 200, 250, or 300 amino acids; and a second CRISPR-Cas effector polypeptide having a length of about 100, 200, 300, 400, 500, 600, 700, 800, 850, or 900 amino acids to about 950, 1,000, 1,050, or 1,100 amino acids.

In some embodiments, the heterologous polypeptide comprises a nuclease domain or a portion thereof, which can be referred to herein as a “heterologous nuclease domain or a portion thereof” since the nuclease domain or a portion thereof from the heterologous polypeptide is heterologous to one or more CRISPR-Cas effector polypeptide(s) present in the engineered protein. The heterologous polypeptide may be a DNA nuclease domain or a portion thereof. In some embodiments, the heterologous nuclease domain or a portion thereof is from a CRISPR-Cas effector protein. In some embodiments, the heterologous nuclease domain or a portion thereof is not from a CRISPR-Cas effector protein. In some embodiments, the heterologous nuclease domain or a portion thereof is from a bacterial protein, optionally wherein the heterologous nuclease domain or a portion thereof is from a restriction endonuclease, homing endonuclease, colicin, pyocin, reverse transcriptase, DNase, and/or a standalone HNH domain. In some embodiments, an engineered protein comprises a heterologous polypeptide that includes a nuclease domain or a portion thereof (i.e., a heterologous nuclease domain or a portion thereof), and the engineered protein is a nuclease that optionally cleaves the target strand of a target nucleic acid and/or the non-target stand of a target nucleic acid. In some embodiments, the engineered protein cleaves the target strand of a target nucleic acid and the non-target stand of the target nucleic acid and provides either a blunt-ended double strand break of the target nucleic acid or a staggered double-strand break of the target nucleic acid. In some embodiments, the engineered protein cleaves the target strand of a target nucleic acid and the non-target stand of the target nucleic acid and the distance (e.g., number of nucleotides) between the cut sites is 0, 1, 2, 3, 4, or 5 nucleotides to about 6, 7, 8, 9, 10, 11, 12, 13, 14, 15, 16, 17, 18, 19, or 20 nucleotides.

In some embodiments, the heterologous nuclease domain or a portion thereof may be a target strand nickase domain or a portion thereof. A “target strand nickase domain or a portion thereof” as used herein refers to a polypeptide that has nickase activity to the target strand of a target nucleic acid when the domain or portion thereof is in its native protein. That is, a target strand nickase domain or a portion thereof can or is capable of nicking (e.g., cleaving or breaking) the target strand (also referred to as the sense (e.g., “+”; template) strand) of the target nucleic acid when the domain or portion thereof is in its native protein. For example, the HNH domain of Cas9 nicks and/or has nickase activity to the target strand of a target nucleic acid. “Nickase activity” as used herein refers to a single-strand break in a nucleic acid.

In some embodiments, a target strand nickase domain or a portion thereof, when present in an engineered protein, may have nickase activity to the target strand of a target nucleic acid. In some embodiments, a target strand nickase domain or a portion thereof, when present in an engineered protein, may have nickase activity to the nontarget strand (also referred to as the antisense (e.g., “−”, complementary) strand) of a target nucleic acid. When a target strand nickase domain or a portion thereof in an engineered protein has nickase activity to both the target strand and nontarget strand, the target strand nickase domain or a portion thereof may cleave both strands sequentially. In some embodiments, a target strand nickase domain or a portion thereof in an engineered protein has more activity (e.g., enzymatic activity) towards the target strand than the nontarget strand of a target nucleic acid. For example, when present in an engineered protein, the target strand nickase domain or a portion thereof may prefer or cleave faster the target strand of a target nucleic acid than the nontarget strand of the target nucleic acid.

A “target strand specific nickase domain” as used herein refers to a polypeptide that has nickase activity only to the target strand of a target nucleic acid and does not nick the nontarget strand of the target nucleic acid. A “nontarget strand specific nickase domain” as used herein refers to a polypeptide that has nickase activity only to the nontarget strand of a target nucleic acid and does not nick the target strand of the target nucleic acid. A “target and nontarget strand nickase domain” as used herein refers to a polypeptide that has nickase activity to both the target strand and the nontarget strand of a target nucleic acid. In some embodiments, an engineered protein comprises a target strand nickase domain or a portion thereof and the target strand nickase domain or a portion thereof is a target strand specific nickase domain in the engineered protein. In some embodiments, an engineered protein comprises a target strand nickase domain or a portion thereof and the target strand nickase domain or a portion thereof is a nontarget strand specific nickase domain in the engineered protein. In some embodiments, an engineered protein comprises a target strand nickase domain or a portion thereof and the target strand nickase domain or a portion thereof is a target and nontarget strand nickase domain in the engineered protein.

An engineered protein may comprise a heterologous polypeptide that comprises target strand nickase domain or a portion thereof. Accordingly, the engineered protein may have nickase activity to the target strand of a target nucleic acid and/or to the nontarget strand of the target nucleic acid. Thereby, the engineered protein may be a target strand nickase and/or a nontarget strand nickase. A “target strand nickase” as used herein in reference to an engineered protein refers to an engineered protein that can or is capable of cleaving the target strand of a target nucleic acid. A “nontarget strand nickase” as used herein in reference to an engineered protein refers to an engineered protein that can or is capable of cleaving the nontarget strand of a target nucleic acid. A “target and nontarget strand nickase” as used herein in reference to an engineered protein refers to an engineered protein that can or is capable of cleaving both the target and nontarget strand of a target nucleic acid in any order (e.g., sequentially or simultaneously). In some embodiments, the engineered protein is a target strand nickase and/or has nickase activity to the target strand of a target nucleic acid. In some embodiments, the engineered protein is a nontarget strand nickase and/or has nickase activity to the nontarget strand of a target nucleic acid. In some embodiments, the engineered protein is a target and nontarget strand nickase and/or has nickase activity to the target strand and nontarget strand of a target nucleic acid.

In some embodiments, the heterologous polypeptide of an engineered protein comprises a target strand nickase domain or a portion thereof and the target strand nickase domain or a portion thereof of the engineered protein has nickase activity to the target strand of a target nucleic acid, thereby the engineered protein is a target strand nickase. In some embodiments, the heterologous polypeptide of an engineered protein comprises a target strand nickase domain or a portion thereof and the target strand nickase domain or a portion thereof of the engineered protein has nickase activity to the nontarget strand of a target nucleic acid, thereby the engineered protein is a nontarget strand nickase. In some embodiments, the heterologous polypeptide of an engineered protein comprises a target strand nickase domain or a portion thereof and the target strand nickase domain or a portion thereof of the engineered protein has nickase activity to the both the target and nontarget strand of a target nucleic acid, thereby the engineered protein is a target and nontarget strand nickase. In some embodiments, the heterologous polypeptide of an engineered protein comprises a target strand nickase domain or a portion thereof and the target strand nickase domain or a portion thereof of the engineered protein has nickase activity to at least the target strand of a target nucleic acid and a CRISPR-Cas effector polypeptide of the engineered protein comprises a nuclease domain or a portion thereof that has nickase activity to at least the nontarget strand of the target nucleic acid, thereby the engineered protein is a target and nontarget strand nickase. In some embodiments, the CRISPR-Cas effector polypeptide of the engineered protein comprises a nuclease domain or portion thereof that is a target and nontarget strand nickase domain or portion thereof, but the nuclease domain or portion thereof is inactivated so that nuclease activity to the target strand is inactivated, thereby the target strand of the target nucleic acid is not nicked by the nuclease domain or portion thereof.

In some embodiments, an engineered protein may comprise one or more (e.g., 1, 2, or more) nuclease domain(s) or a portion thereof. In some embodiments, an engineered protein comprises at least two different nuclease domains or a portion thereof. In some embodiments, an engineered protein may comprise a native nuclease domain, optionally one or more (e.g., 1, 2, or more) native nuclease domain(s). A “native nuclease domain” as used herein refers to a nuclease domain that is naturally present in a CRISPR-Cas effector protein. In some embodiments, an engineered protein comprises a first heterologous nuclease domain (e.g., from and/or present in the heterologous polypeptide) and a second nuclease domain. The second nuclease domain may be from and/or present in CRISPR-Cas effector protein. In some embodiments, the first nuclease domain may be a native nuclease domain and/or the second nuclease domain may be a native nuclease domain. In some embodiments, the second nuclease domain is a target and nontarget strand nickase domain or a portion thereof. A “nontarget and target strand nickase domain or a portion thereof” as used herein refers to a polypeptide that has nickase activity to the nontarget strand of a target nucleic acid and to the target strand of the target nucleic acid when the domain or portion thereof is in its native protein, and cleaves the nontarget strand before the target strand or prefers or cleaves faster the nontarget strand than the target strand. A nontarget and target strand nickase domain or a portion thereof may provide a staggered double strand break in the target nucleic acid. In some embodiments, the second nuclease domain is active. In some embodiments, the second nuclease domain is deactivated (i.e., dead, inactive, or devoid of nickase activity). In some embodiments, the second nuclease domain only nicks the nontarget strand of a target nucleic acid and/or comprises a mutation that inactivates nickase activity to the target strand of a target nucleic acid. A nuclease domain or portion thereof in an engineered protein may be deactivated by a mutation in the nuclease domain or portion thereof that removes or inactivates nickase activity. In some embodiments, an engineered protein comprises a nuclease domain or portion thereof from a Type V CRISPR-Cas effector protein such as a Cas12a (e.g., from one of SEQ ID NO:50-66) or Cas12b (e.g., from SEQ ID NO:151). In some embodiments, the nuclease domain is a RuvC domain from a Type V CRISPR-Cas effector protein such as a Cas12a or Cas12b. An engineered protein may comprise one or more nuclease domain(s) that provide a blunt-ended double strand break of a target nucleic acid or a staggered double-strand break of a target nucleic acid.

In some embodiments, the heterologous polypeptide of an engineered protein comprises all or a portion of a HNH domain of a CRISPR-Cas effector protein. The heterologous polypeptide and/or HNH domain may comprise and/or form a zinc finger motif. In some embodiments, the heterologous polypeptide and/or HNH domain has a length of about 10, 20, 30, 40, 50, 60, 70, 80, 90, or 100 amino acids to about 110, 125, 150, 175, 200, 225, 250, 275, or 300 amino acids. The heterologous polypeptide and/or HNH domain may comprise about 25 or 30 to about 40 or 45 amino acids and/or may comprise one or at least two histidines and an asparagine that are optionally in a nucleic acid binding and cleavage site. In some embodiments, the heterologous polypeptide and/or HNH domain may comprise about 25 or 30 to about 40 or 45 amino acids that include two histidines and one asparagine that are present in and/or form a zinc finger motif. The heterologous polypeptide and/or HNH domain may comprise and/or form two antiparallel beta-strands that are linked by a loop and/or may comprise an alpha helix, optionally wherein a histidine is present in at least one of the beta-strands, an asparagine is present in the loop, and/or a histidine or asparagine is present in the alpha-helix. The heterologous polypeptide may comprise all or a portion of a HNH domain having a structure as described in Pediaditakis M, et al. Journal of Bacteriology 194(22); 6184-6194. In some embodiments, the heterologous polypeptide of an engineered protein comprises all or a portion of a HNH domain of a Type II CRISPR-Cas effector protein such as a Cas9 HNH domain. The heterologous polypeptide of an engineered protein may comprise all or a portion of a HNH domain (e.g., a Cas9 HNH domain) that is inactive. The HNH domain or a portion thereof may have an inactivating mutation (e.g., a mutation that removes nickase activity). In some embodiments, the heterologous polypeptide of an engineered protein comprises all or a portion of an HNH domain that has an inactivating mutation and/or the HNH domain is inactive (e.g., does not have nickase activity). In some embodiments, the heterologous polypeptide of an engineered protein comprises all or a portion of an inactivated HNH domain that has a H840A mutation. In some embodiments, the heterologous polypeptide of an engineered protein comprises an amino acid sequence having at least 70%, 75%, 80%, 85%, 90%, 95%, 96%, 97%, 98%, 99%, or 100% sequence identity to the amino acid sequence of one or more of SEQ ID NOs:1 or 169-174. In some embodiments, the heterologous polypeptide of an engineered protein comprises the amino acid sequence of any one of SEQ ID NOs:1 or 169-174.

In some embodiments, the heterologous polypeptide of an engineered protein comprises an amino acid sequence that has an amino acid residue that is not a histidine residue at a position corresponding to amino number 839 of SEQ ID NO:81, when the amino acid sequence of the heterologous polypeptide and SEQ ID NO:81 are optimally aligned. In some embodiments, the heterologous polypeptide of an engineered protein comprises an amino acid sequence that has an amino acid residue that is not a histidine residue at a position corresponding to amino number 75 of SEQ ID NO:1, when the amino acid sequence of the heterologous polypeptide and SEQ ID NO:1 are optimally aligned. In some embodiments, the heterologous polypeptide of an engineered protein comprises an amino acid sequence that has an alanine residue at a position corresponding to amino number 839 of SEQ ID NO:81, when the amino acid sequence of the heterologous polypeptide and SEQ ID NO:81 are optimally aligned. In some embodiments, the heterologous polypeptide of an engineered protein comprises an amino acid sequence that has an alanine residue at a position corresponding to amino number 75 of SEQ ID NO:1, when the amino acid sequence of the heterologous polypeptide and SEQ ID NO:1 are optimally aligned.

In some embodiments, the heterologous polypeptide of an engineered protein may be between and/or linked to (e.g., directly or indirectly) two consecutive or nonconsecutive amino acids that are present in a CRISPR-Cas effector protein. In some embodiments, the engineered protein is prepared by inserting a heterologous polypeptide between two consecutive or nonconsecutive amino acids of a CRISPR-Cas effector protein or a portion thereof. In some embodiments, an engineered protein may comprise in the amino terminal to carboxy terminal direction, a first CRISPR-Cas effector polypeptide, a heterologous polypeptide, and a second CRISPR-Cas effector polypeptide, with the first and second CRISPR-Cas effector polypeptides being from the same CRISPR-Cas effector protein.

In some embodiments, a CRISPR-Cas effector polypeptide comprises a portion of a Type V CRISPR-Cas effector protein such as Cas12a or Cas12b. The CRISPR-Cas effector polypeptide may comprise all or a portion of a nucleic acid binding domain such as a nucleic acid binding domain from a Type V CRISPR-Cas effector protein (e.g., Cas12a or Cas12b). In some embodiments, a CRISPR-Cas effector polypeptide of an engineered protein comprises an amino acid sequence having at least 70%, 75%, 80%, 85%, 90%, 95%, 96%, 97%, 98%, 99%, or 100% sequence identity to a portion of the amino acid sequence of one or more of SEQ ID NOs:50-66 or 151. In some embodiments, a CRISPR-Cas effector polypeptide comprises a portion of the amino acid sequence of any one of SEQ ID NOs:50-66 or 151. In some embodiments, an engineered protein comprises two or more (e.g., 2, 3, 4, or more) separate portions of the amino acid sequence of any one of SEQ ID NOs:50-66 or 151.

In some embodiments, an engineered protein of the present invention may be devoid of about 5, 10, 15, 20, 25, 30, 35, 40, 45, 50, 55, 60, 65, 70, 75, 80, 85, 90, 95, 100 or more amino acids that are present in a CRISPR-Cas effector protein such as one having a sequence of any one of SEQ ID NOs:50-66 or 151. In some embodiments, an engineered protein of the present invention may be devoid of 0, 1, 2, 3, 4, 5, 6, 7, 8, 9, 10, 11, 12, 13, 14, or 15 amino acids that are present in a CRISPR-Cas effector protein such as one having a sequence of any one of SEQ ID NOs:50-66 or 151. For example, an engineered protein may be devoid of one or more amino acids from amino acid residue 283 to amino acid residue 293 of SEQ ID NO:50 or SEQ ID NO:58; from amino acid residue 331 to amino acid residue 341 of SEQ ID NO:55; from amino acid residue 312 to amino acid residue 322 of SEQ ID NO:51; or from corresponding amino acid residues for a sequence that is optimally aligned to one of SEQ ID NOs:50, 51, 58, or 55 (e.g., from amino acid residues that correspond to amino acid residues 283-293 when a sequence (e.g., SEQ ID NO:52) is optimally aligned to SEQ ID NO:50). In some embodiments, an engineered protein is devoid of one or more (e.g., 1, 2, 3, 4, or more) interdomain linker region(s) (e.g., a region that is between two domains such as two adjacent domains) that are present in a CRISPR-Cas effector protein (e.g., one having a sequence of any one of SEQ ID NOs:50-66 or 151) from which a CRISPR-Cas effector is a portion of and that is present in the engineered protein.

In some embodiments, the heterologous polypeptide of an engineered protein may be between and/or linked to (e.g., directly or indirectly) two consecutive or nonconsecutive amino acids of a CRISPR-Cas effector protein (e.g., a CRISPR-Cas effector protein having an amino acid sequence of any one of SEQ ID NOs:50-66 or 151). For example, an engineered protein may comprise, from the N- to C-terminus, a first CRISPR-Cas effector polypeptide, an HNH domain, and a second CRISPR-Cas effector polypeptide, wherein the first and second CRISPR-Cas effector polypeptides are each a portion of a CRISPR-Cas effector protein and the last amino acid residue at the C-terminus of the first CRISPR-Cas effector polypeptide and the first amino acid reside at the N-terminus of the second CRISPR-Cas effector polypeptide are two consecutive or nonconsecutive amino acid residues of the a CRISPR-Cas effector protein. The heterologous polypeptide may be linked directly to one or both of the two consecutive or nonconsecutive amino acids of the CRISPR-Cas effector protein (i.e., no linker is used to attach one terminus of the heterologous polypeptide to a terminus of a CRISPR-Cas effector polypeptide that is a portion of the CRISPR-Cas effector protein). In some embodiments, the heterologous polypeptide may be linked indirectly (e.g., via a linker such as a peptide linker) to one or both of the two consecutive or nonconsecutive amino acids of the CRISPR-Cas effector protein. In some embodiments, the heterologous polypeptide of an engineered protein may be between and/or linked to (e.g., directly or indirectly) two consecutive amino acids of a CRISPR-Cas effector protein (e.g., a CRISPR-Cas effector protein having an amino acid sequence of any one of SEQ ID NOs:50-66 or 151). In some embodiments, the heterologous polypeptide of an engineered protein may be between and/or linked to (e.g., directly or indirectly) two nonconsecutive amino acids of a CRISPR-Cas effector protein (e.g., a CRISPR-Cas effector protein having an amino acid sequence of any one of SEQ ID NOs:50-66 or 151).

In some embodiments, the two consecutive or nonconsecutive amino acids are two of the amino acid residues from amino acid residue 250, 260, 270, or 280 to amino acid residue 290, 300, 310, 320, 330, 340, or 350 that are consecutive or nonconsecutive, respectively. In some embodiments, the heterologous polypeptide may be between and/or linked to (e.g., directly or indirectly) two consecutive or nonconsecutive amino acids that are two of the following amino acid residues: amino acid residues 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, and 350 of a CRISPR-Cas effector protein (e.g., a CRISPR-Cas effector protein having an amino acid sequence of any one of SEQ ID NOs:50-66 or 151). In some embodiments, the heterologous polypeptide of an engineered protein may be between and/or linked to (e.g., directly or indirectly) two nonconsecutive amino acids of a CRISPR-Cas effector protein (e.g., a CRISPR-Cas effector protein having an amino acid sequence of any one of SEQ ID NOs:50-66 or 151), wherein one of the two nonconsecutive amino acid residues is amino acid residue 270, 271, 272, 273, 274, 275, 276, 277, 278, 279, 280, 281, 282, 283, 284, or 285 and the other of the two nonconsecutive amino acid residues is amino acid residue 286, 287, 288, 289, 290, 291, 292, 293, 294, 295, 296, 297, 298, 299, 300, 301, 302, 303, 304, or 305, optionally of SEQ ID NOs:50-66 or 151. In some embodiments, the heterologous polypeptide is between and/or linked to (e.g., directly or indirectly) amino acid residues 290 and 291, amino acid residues 291 and 292, amino acid residues 292 and 293, amino acid residues 293 and 294, amino acid residues 320 and 321, amino acid residues 321 and 322, amino acid residues 339 and 340, or amino acid residues 340 and 341 of a CRISPR-Cas effector protein such as a CRISPR-Cas effector protein having an amino acid sequence of any one of SEQ ID NOs:50-66 or 151. For example, in some embodiments, the heterologous polypeptide may be between and/or linked to (e.g., directly or indirectly) amino acid residues 290 and 291 of SEQ ID NO:50; amino acid residues 291 and 292 of SEQ ID NO:50; amino acid residues 291 and 292 of SEQ ID NO:58; amino acid residues 292 and 293 of SEQ ID NO:58; amino acid residues 320 and 321 of SEQ ID NO:51; amino acid residues 321 and 322 of SEQ ID NO:51; amino acid residues 322 and 323 of SEQ ID NO:51; amino acid residues 339 and 340 of SEQ ID NO:55; amino acid residues 340 and 341 of SEQ ID NO:55; or corresponding amino acid residues for a sequence that is optimally aligned to one of SEQ ID NOs:50, 51, 58, or 55 (e.g., amino acid residues that correspond to amino acid residues 291 and 292 when a sequence (e.g., SEQ ID NO:52) is optimally aligned to SEQ ID NO:50). In some embodiments, the heterologous polypeptide of an engineered protein may be in an interdomain linker region (e.g., a region that is between two domains such as two adjacent domains) of a CRISPR-Cas effector protein. In some embodiments, the heterologous polypeptide may be positioned in an engineered protein such that it is adjacent to an exposed portion of the target strand of a target nucleic acid.

In some embodiments, an engineered protein comprises all or a portion of a wedge domain, a Rec1 domain, a Rec2 domain, a PAM-interacting domain, a RuvC domain, a bridge helix, and/or a Nuc domain each of which may be from a Type V CRISPR-Cas effector protein such as Cas12a, Cas12b, and/or a protein having a sequence of any one of SEQ ID NOs:50-66 or 151. In some embodiments, an engineered protein comprises all or a portion of a Cas12a domain having a structure as described in Yamano, Takashi, et al., Mol Cell 67: 633-645 (2017). In some embodiments, the heterologous polypeptide of an engineered protein may be between a polypeptide for all or a portion of a Rec1 domain and a polypeptide for all or a portion of a Rec2 domain each of which may be from a Type V CRISPR-Cas effector protein such as Cas12a, Cas12b, and/or a protein having a sequence of any one of SEQ ID NOs:50-66 or 151. In some embodiments, all or a portion of the heterologous polypeptide is at an exposed surface or interface of the engineered protein. In some embodiments, the CRISPR-Cas effector polypeptide of an engineered protein comprises all or a portion of a RuvC domain. As one of skill in the art would understand, some domains (e.g., the wedge and RuvC domains of Cas12a) are not continuous in sequence and may be split into two or more (e.g., 2, 3, 4, or more) non-continuous sequences. For example, the polypeptide for Cas12 may have the following from the N- to C-terminus: first portion of the wedge domain (WED-1), Rec1 domain, Rec2 domain, second portion of the wedge domain (WED-2), PAM-interacting domain (PI), third portion of the wedge domain (WED-3), first portion of the RuvC domain (RuvC-1), bridge helix, second portion of the RuvC domain (RuvC-2), Nuc domain, and third portion of the RuvC domain (RuvC-3). In some embodiments, an engineered protein comprises all or a portion of an active RuvC domain. In some embodiments, an engineered protein comprises all or a portion of an inactivated RuvC domain, optionally all or a portion of an inactivated RuvC domain that has a D10A mutation. In some embodiments, an engineered protein comprises all or a portion of an inactivated RuvC domain and the polypeptide comprising all or a portion of the inactivated RuvC domain has an alanine at a position corresponding to amino acid residue 831 SEQ ID NO:50 when the polypeptide is optimally aligned to SEQ ID NO:50, optionally wherein the mutation is referred to as a D10A and/or D832A mutation.

A CRISPR-Cas effector polypeptide may comprise a nuclease, optionally a RuvC like nuclease. In some embodiments, a CRISPR-Cas effector polypeptide comprises a RuvC domain or a portion thereof. In some embodiments, a CRISPR-Cas effector polypeptide comprises a nuclease in the Rnase H superfamily. In some embodiments, a CRISPR-Cas effector polypeptide comprises RNase H-like enzyme having a catalytic core that may include a β-sheet comprising five β-strands, ordered 32 145, optionally where the β-strand 2 is antiparallel to the other β-strands. On both sides the central β-sheet may be flanked by α-helices, the number of which may differ between related enzymes. In some embodiments, a CRISPR-Cas effector polypeptide comprises a RNase H-like catalytic core where the active site residues include one or more of aspartic acid, glutamic acid and histidine. In some embodiments, a CRISPR-Cas effector polypeptide comprising a RNase H-like catalytic core may include negatively charged side chains in the active sites of the RNase H-like polypeptide that, directly or through a water molecule, are involved in coordinating a divalent metal ion. In some embodiments, a CRISPR-Cas effector polypeptide comprises RNase H-like catalytic core that uses a two ion-dependent mechanism of catalysis, optionally wherein the ion is Mg²⁺ and/or Mn²⁺. In some embodiments, a CRISPR-Cas effector polypeptide comprises nuclease and/or RNase H-like nuclease as described in Majorek K A, et al. Nucleic Acids Res. 2014; 42(7):4160-4179, which is incorporated herein by reference in its entirety.

In some embodiments, a CRISPR-Cas effector polypeptide comprises one or more (e.g., 1, 2, 3, 4 or more) mutations. The one or more mutations may be to improve or modify the activity of a heterologous polypeptide and/or the activity of a CRISPR-Cas effector polypeptide. In some embodiments, a CRISPR-Cas effector polypeptide may comprise an inactivating mutation such as a D10A mutation in the RuvC domain. In some embodiments, a CRISPR-Cas effector polypeptide comprises all or a portion of a Rec1 domain that comprises one or more (e.g., 1, 2, 3, 4 or more) mutations such as in one or more of amino acid residue(s) 243-253 of a CRISPR-Cas effector protein (e.g., a CRISPR-Cas effector protein having an amino acid sequence of any one of SEQ ID NOs:50-66 or 151) and/or in the sequence GFVTESGEKIK (SEQ ID NO:122). In some embodiments, a CRISPR-Cas effector polypeptide comprises a hairpin and/or the sequence GFVTESGEKIK (SEQ ID NO:122), and one or more of the amino acid residue(s) in the hairpin and/or sequence may be mutated. In some embodiments, a CRISPR-Cas effector polypeptide comprises a hairpin and/or the sequence GFVTESGEKIK (SEQ ID NO:122), and all or a portion of the hairpin and/or sequence is deleted. In some embodiments, a CRISPR-Cas effector polypeptide comprises a hairpin and/or the sequence GFVTESGEKIK (SEQ ID NO:122), and 1, 2, 3, 4, 5, or more amino acid residues are added to one or both ends of the hairpin and/or sequence.

In some embodiments, an engineered protein comprises, from the N- to C-terminus, a first CRISPR-Cas effector polypeptide, an HNH domain, and a second CRISPR-Cas effector polypeptide, wherein the first and second CRISPR-Cas effector polypeptides are each a portion of deactivated LbCas12a (e.g., LbCas12a having a sequence of SEQ ID NO:50) and the last amino acid residue at the C-terminus of the first CRISPR-Cas effector polypeptide and the first amino acid reside at the N-terminus of the second CRISPR-Cas effector polypeptide are two consecutive amino acid residues of the deactivated LbCas12a. The HNH domain may be from Streptococcus pyogenes Cas9 (SpCas9) and/or may have a sequence comprising SEQ ID NO:1. In some embodiments, the HNH domain may have a sequence of any one of SEQ ID NOs:1 or 169-174. The HNH domain may be positioned in the engineered protein such that it is adjacent to an exposed portion of the target strand of a target nucleic acid. The engineered protein may be a target strand nickase. In some embodiments, the engineered protein only nicks the target DNA strand. In some embodiments, the engineered protein is a target and nontarget strand nickase.

One or more (e.g., 1, 2, 3, 4, or more) linker(s) may be present in an engineered protein. For example, a linker may be present between a CRISPR-Cas effector polypeptide and a heterologous polypeptide. In some embodiments, a linker may be present between a first CRISPR-Cas effector polypeptide and a heterologous polypeptide and a linker may be present between the heterologous polypeptide and a second CRISPR-Cas effector polypeptide. Exemplary linkers include, but are not limited to, those described herein. In some embodiments, the linker comprises 1 to 2, 3, 4, 5, 6, 7, 8, 9, or 10 amino acids and/or comprises glycine and/or serine. In some embodiments, the linker comprises 1, 2, 3, or 4 amino acids that are glycine and/or serine. In some embodiments, the engineered protein is devoid of a linker between a CRISPR-Cas effector polypeptide and a heterologous polypeptide. In some embodiments, the heterologous polypeptide is indirectly linked to an amino acid residue at the N-terminus of a CRISPR-Cas effector polypeptide via a linker and/or the heterologous polypeptide is indirectly linked to an amino acid residue at the C-terminus of a CRISPR-Cas effector polypeptide via a linker.

In some embodiments, the heterologous polypeptide is directly linked (i.e., without a linker) to an amino acid residue at the N-terminus of a CRISPR-Cas effector polypeptide and/or the heterologous polypeptide is directly linked (i.e., without a linker) to an amino acid residue at the C-terminus of a CRISPR-Cas effector polypeptide.

An engineered protein may comprise an amino acid sequence having at least 70%, 75%, 80%, 85%, 90%, 95%, 96%, 97%, 98%, 99%, or more sequence identity to any one of SEQ ID NOs:2-17, 125-132, or 157-168. In some embodiments, an engineered protein comprises and/or has an amino acid sequence of any one of SEQ ID NOs:2-17, 125-132, or 157-168. An engineered protein may have at least 70%, 75%, 80%, 85%, 90%, 95%, 96%, 97%, 98%, 99%, or more sequence identity to all or a portion of an amino acid sequence of a wild-type CRISPR-Cas effector protein. In some embodiments, the engineered protein has at least 70%, 75%, 80%, 85%, 90%, 95%, 96%, 97%, 98%, 99%, or more sequence identity to all or a portion of the amino acid sequence of any one of SEQ ID NOs:50-66 or 151. In some embodiments, the engineered protein has about 70%, 75%, or 80% to about 85%, 90%, 95%, or 98% sequence identity to all or a portion of the amino acid sequence of any one of SEQ ID NOs:50-66 or 151.

An engineered protein may have increased efficiency compared to a CRISPR-Cas effector protein (e.g., Cas12a, a CRISPR-Cas effector protein having a sequence of SEQ ID NOs:50-66 or 151, and/or a wild-type CRISPR-Cas effector protein) such as increased efficiency in nicking the target strand and/or nontarget strand of a target nucleic acid. In some embodiments, an engineered protein may have increased efficiency compared to a CRISPR-Cas effector protein (e.g., Cas12a, a CRISPR-Cas effector protein having a sequence of SEQ ID NOs:50-66 or 151, and/or a wild-type CRISPR-Cas effector protein) in nicking the target strand of a target nucleic acid. In some embodiments, an engineered protein may provide for an increased number of target strand breaks in a target nucleic acid compared to the number of target strand breaks in the target nucleic acid with a CRISPR-Cas effector protein (e.g., Cas12a, a CRISPR-Cas effector protein having a sequence of SEQ ID NOs:50-66 or 151, and/or a wild-type CRISPR-Cas effector protein). In some embodiments, an engineered protein may have increased efficiency compared to a CRISPR-Cas effector protein (e.g., Cas12a, a CRISPR-Cas effector protein having a sequence of SEQ ID NOs:50-66 or 151, and/or a wild-type CRISPR-Cas effector protein) in modifying a target nucleic acid.

Compositions, complexes, and systems comprising an engineered protein may be provided according to embodiments of the present invention. In some embodiments, a composition, complex and/or system comprising an engineered protein may be a base editing composition, complex, and/or system. A composition, complex, and/or system of the present invention may include a guide nucleic acid (e.g., a guide RNA) and/or a deaminase (e.g., a cytosine deaminase and/or an adenine deaminase). In some embodiments, an engineered protein, guide nucleic acid, and optionally deaminase form a complex or are comprised in a complex (e.g., a ribonucleoprotein). The engineered protein, guide nucleic acid, and optionally deaminase may not naturally occur together and/or a complex comprising the engineered protein, guide nucleic acid, and optionally deaminase may not naturally occur together. In some embodiments, an engineered protein comprises and/or is fused to a deaminase (e.g., an adenine deaminase and/or a cytosine deaminase).

Also provided herein are nucleic acid molecules encoding an engineered protein of present invention along with an expression cassettes and/or vector comprising a nucleic acid molecule of the present invention.

According to some embodiments, a method is provided that comprises contacting a target nucleic acid with: an engineered protein of the present invention, a guide nucleic acid (e.g., a guide RNA), and optionally a deaminase. In some embodiments, the engineered protein, the guide nucleic acid, and/or deaminase form a complex or are comprised in a complex. In some embodiments, the method may modify the target nucleic acid and/or may provide one or more single strand breaks in the target nucleic acid.

In some embodiments, a composition, system, method, and/or complex comprising an engineered protein may have increased efficiency compared to a composition, system, method, and/or complex comprising a CRISPR-Cas effector protein (e.g., Cas12a, a CRISPR-Cas effector protein having a sequence of SEQ ID NOs:50-66 or 151, and/or a wild-type CRISPR-Cas effector protein). In some embodiments, a composition, system, method, and/or complex comprising an engineered protein that is a target strand nickase may have increased efficiency compared to a composition, system, method, and/or complex comprising a CRISPR-Cas effector protein (e.g., Cas12a, a CRISPR-Cas effector protein having a sequence of SEQ ID NOs:50-66 or 151, and/or a wild-type CRISPR-Cas effector protein). This may be because nicking the target strand may increase the efficiency of genome editing tools such as base editors and/or base diversifiers. In some embodiments, a composition, system, method, and/or complex comprising an engineered protein may provide for an increased number of target strand breaks in a target nucleic acid compared to the number of target strand breaks in the target nucleic acid with a composition, system, method, and/or complex comprising a CRISPR-Cas effector protein (e.g., Cas12a, a CRISPR-Cas effector protein having a sequence of SEQ ID NOs:50-66 or 151, and/or a wild-type CRISPR-Cas effector protein).

An engineered protein and/or a composition, system, method, and/or complex comprising an engineered protein may provide improved or altered indel size and/or composition, improved or altered deletion size in a target nucleic acid, improved or altered nicking ability on either strand (i.e., target or nontarget strand of a target nucleic acid), and/or increased nuclease activity compared to a CRISPR-Cas effector protein (e.g., Cas12a, a CRISPR-Cas effector protein having a sequence of SEQ ID NOs:50-66 or 151, and/or a wild-type CRISPR-Cas effector protein) and/or to a composition, system, method, and/or complex comprising a CRISPR-Cas effector protein (e.g., Cas12a, a CRISPR-Cas effector protein having a sequence of SEQ ID NOs:50-66 or 151, and/or a wild-type CRISPR-Cas effector protein). In some embodiments, an engineered protein and/or a composition, system, method, and/or complex comprising an engineered protein imparts nuclease function onto a catalytically inactivated CRISPR-Cas effector protein. In some embodiments, an engineered protein and/or a composition, system, method, and/or complex comprising an engineered protein provides a different editing profile and/or a different cleavage pattern for a target nucleic acid compared to the editing profile and/or a different cleavage pattern of the target nucleic acid for a Cas effector protein ((e.g., Cas12a, a CRISPR-Cas effector protein having a sequence of SEQ ID NOs:50-66 or 151, and/or a wild-type CRISPR-Cas effector protein) and/or a composition, system, method, and/or complex comprising a CRISPR-Cas effector protein (e.g., Cas12a, a CRISPR-Cas effector protein having a sequence of SEQ ID NOs:50-66 or 151, and/or a wild-type CRISPR-Cas effector protein).

In some embodiments, a method of the present invention may have increased efficiency in modifying a target nucleic acid compared to the efficiency of a control method (e.g., a method comprising contacting the target nucleic acid with a CRISPR-Cas effector protein (e.g., Cas12a, a CRISPR-Cas effector protein having a sequence of SEQ ID NOs:50-66 or 151, and/or a wild-type CRISPR-Cas effector protein) and/or that is devoid of an engineered protein).

As described herein, the engineered proteins, nucleic acids, expression cassettes, and/or vectors of the present invention may be codon optimized for expression in an organism. An organism useful with this invention may be any organism or cell thereof for which nucleic acid modification may be useful. An organism can include, but is not limited to, any animal (e.g., a mammal), any plant, any fungus, any archaeon, or any bacterium. In some embodiments, the organism may be a plant or cell thereof. In some embodiments, the organism is an animal such as a mammal (e.g., a human).

The target nucleic acid may be a genomic sequence from any organism (e.g., eukaryote such as a mammal or a plant). In some embodiments, the target nucleic acid is a genomic sequence from a model organism such as, but not limited to, Escherichia coli, an immortalized human cell line (e.g., HEK293, HeLa, etc.), Caenorhabditis elegans, and/or Drosophila Melanogaster. In some embodiments, the target nucleic acid is a genomic sequence from a non-model organism. Exemplary non-model organisms include, but are not limited to crop plants (e.g., fruit crop plants, vegetable crop plants, and/or field crop plants) and/or animals such as humans, primates and/or mice. In some embodiments, the non-model organism is a crop plant such as corn, soybean, wheat, or canola. In some embodiments, the non-model organism is an animal for testing and/or use of a human therapeutic.

A target nucleic acid of any plant or plant part may be modified using the nucleic acid constructs of the invention. Any plant (or groupings of plants, for example, into a genus or higher order classification) may be modified using an engineered protein of the invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry.

In some embodiments, the invention provides cells (e.g., plant cells, animal cells, bacterial cells, archaeon cells, and the like) comprising the polypeptides, polynucleotides, nucleic acid constructs, expression cassettes or vectors of the invention.

The present invention further comprises a kit or kits to carry out the methods of this invention. A kit of this invention can comprise reagents, buffers, and apparatus for mixing, measuring, sorting, labeling, etc., as well as instructions and the like as would be appropriate for modifying a target nucleic acid.

In some embodiments, the invention provides a kit for comprising one or more nucleic acid constructs of the invention, and/or expression cassettes and/or vectors and/or cells comprising the same as described herein, with optional instructions for the use thereof. In some embodiments, a kit may further comprise a CRISPR-Cas guide nucleic acid (corresponding to an engineered protein, which may be encoded by a polynucleotide of the invention) and/or expression cassettes and/or vectors and or cells comprising the same. In some embodiments, a guide nucleic acid may be provided on the same expression cassette and/or vector as one or more nucleic acid constructs of the invention. In some embodiments, the guide nucleic acid may be provided on a separate expression cassette or vector from that comprising the one or more nucleic acid constructs of the invention.

Accordingly, in some embodiments, kits are provided comprising a nucleic acid construct comprising (a) a polynucleotide(s) as provided herein and (b) a promoter that drives expression of the polynucleotide(s) of (a). In some embodiments, the kit may further comprise a nucleic acid construct encoding a guide nucleic acid, wherein the construct comprises a cloning site for cloning of a nucleic acid sequence identical or complementary to a target nucleic acid sequence into backbone of the guide nucleic acid.

In some embodiments, the nucleic acid construct of the invention may be an mRNA that may encode one or more introns within the encoded polynucleotide(s). In some embodiments, the nucleic acid constructs of the invention, and/or an expression cassettes and/or vectors comprising the same, may further encode one or more selectable markers useful for identifying transformants (e.g., a nucleic acid encoding an antibiotic resistance gene, herbicide resistance gene, and the like).

A polypeptide, polynucleotide, nucleic acid construct, expression cassette, vector, composition, kit, system and/or cell of the present invention may comprise all or a portion of a sequence of one or more of SEQ ID NOs:1-175. In some embodiments, a polypeptide, polynucleotide, nucleic acid construct, expression cassette, vector, composition, kit, system and/or cell of the present invention may comprise at least about 20%, 25%, 30%, 35%, 40%, 45%, 50%, 55%, 60%, 65%, 70%, 75%, 80%, 85%, 90%, 95%, 96%, 97%, 98%, 99%, or more consecutive amino acids of a sequence of one or more of SEQ ID NOs:1-175.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

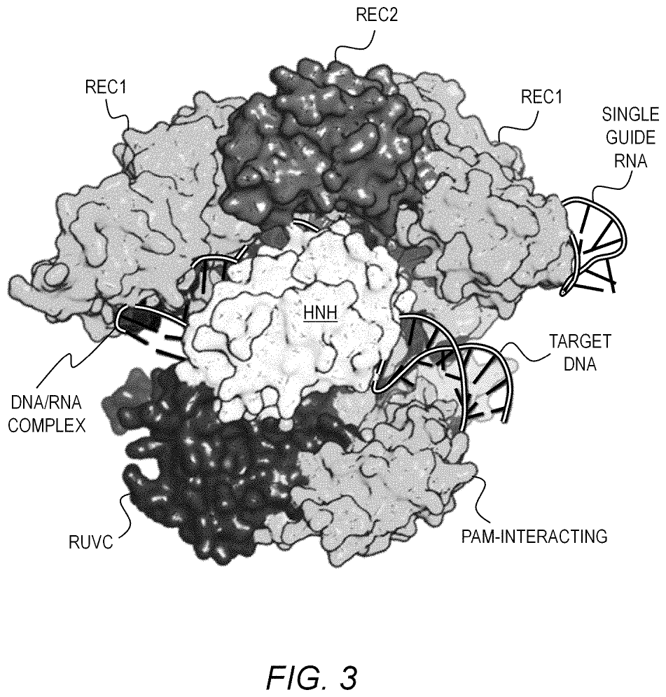

Using existing domain annotations along with visual inspection of the SpCas9 crystal structure (PDB ID 4UN3) in PyMOL (The PyMOL Molecular Graphics System, Version 2.0. Schrodinger, LLC), the full HNH domain from SpCas9 (FIG. 3) was first identified and its residue boundaries determined. The domain is largely resolved in the crystal structure, but several residues connecting the N terminus of the HNH domain to the Rec1 domain are unresolved in the crystal structure. The location of Cas9 target DNA strand cleavage site relative to the HNH domain was also noted. This relative orientation was mimicked in subsequent rational positioning of the HNH domain relative to the target DNA strand of Cas12a.

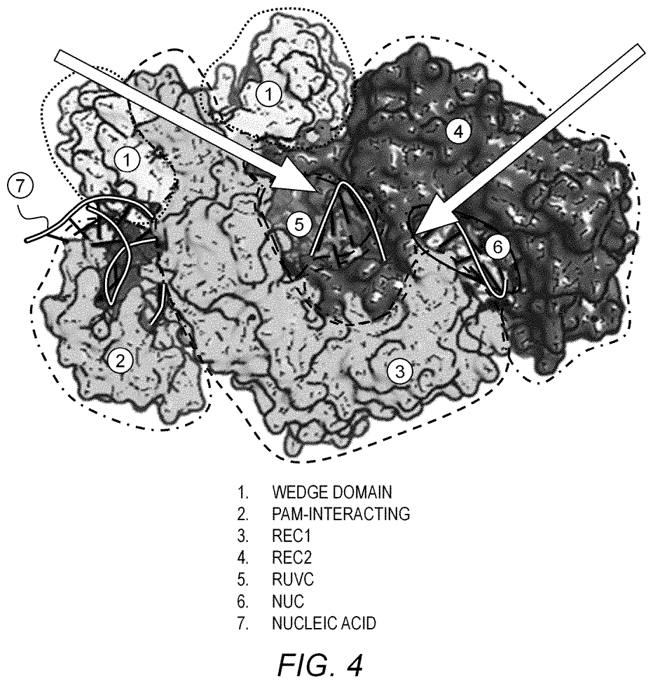

The crystal structure of the LbCas12a ternary complex (PDB ID 5XUS) was next examined to locate an accessible region of the target DNA strand. Although the side of the target DNA/crRNA duplex closest to the RuvC domain is heavily shielded by other Cas12a domains, there is an exposed portion of the target DNA (indicated by the left arrow in FIG. 4) on the opposite side of the protein at the interface between the Rec1 and Rec2 domains (FIG. 4). A linker (indicated by the right arrow in FIG. 4) connecting the two domains sits adjacent to this exposed site and has few interactions with other residues in LbCas12a; this linker was chosen as a candidate site for domain insertion.

To determine the precise placement of the SpCas9 HNH domain relative to LbCas12a, the exposed DNA bases in the groove between the Rec1 and Rec2 domains of LbCas12a were next identified. Then, treating the HNH domain and its target DNA (four bases with two on each side of the cleavage site) as a unit, alignments of the HNH target DNA to the exposed target strand of LbCas12a were tested in a sliding window using PyMOL until an alignment was identified that would place the HNH domain near the insertion loop and would minimize clashes with other domains of LbCas12a. The position of the HNH domain was then adjusted manually using PyMOL to minimize clashes between HNH and Cas12a.

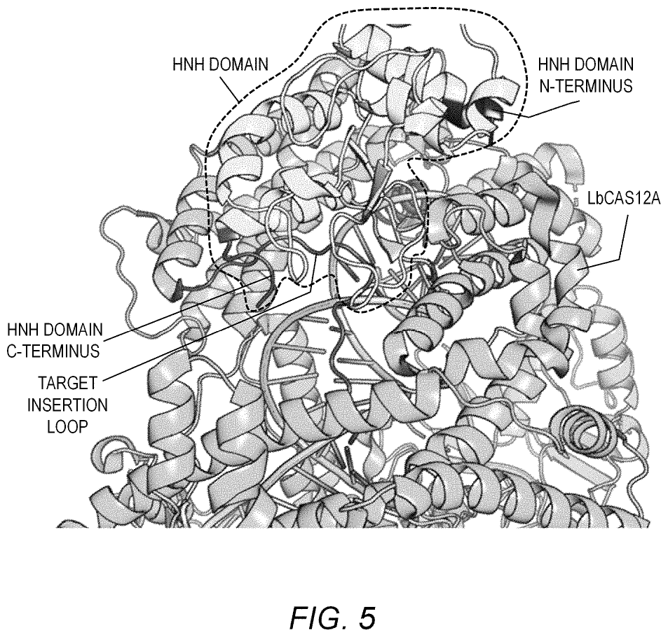

The final selected position of the HNH domain is shown in FIG. 5. While the C terminus of the HNH domain is very close to the C terminal end of the insertion loop, the HNH N terminus is relatively far from the insertion loop; however, this structure does not include the unstructured residues linking the SpCas9 Rec1 and HNH domains. A highly conserved hairpin in this region that interacts with the target DNA/crRNA duplex was further identified as a potential site for later design.

To prepare the Cas12a-HNH fusion structure for computational linker modeling, the N terminus of the HNH domain was initially extended by appending residues from SpCas9 that connect the Rec1 and HNH domains (and which are unresolved in the SpCas9 crystal structure) using PyMOL. The resulting structure was exported and prepared for linker modeling using custom Python scripts that inserted the HNH domain residues into possible insertion sites throughout the insertion loop as shown in Table 1.

TABLE 1
Results of preliminary computational screening
of possible insertion sites in Cas12a.

| | # successful | |
|---|---|---|
| | closures/10 | |
| Insertion Site | attempts | Observations |
| 282 | 0 | |
| 283 | 0 | |
| 284 | 0 | |
| 285 | 3 | |
| 286 | 0 | |
| 287 | 1 | |
| 288 | 3 | When loop closure does occur, it would |
| | | likely offer little flexibility for the |
| | | N-terminal linker. The C-terminal linker |
| | | seems long enough to be flexible. |
| 289 | 2 | |
| 290 | 0 | |
| 291 | 9 | Insertion in a flexible location between |
| | | glycine and glutamate residues |
| 292 | 9 | Insertion in a flexible location between |
| | | glutamate and glycine residues |
| 293 | 6 | Insertion would be immediately before a |
| | | tyrosine that interacts with other |
| | | Cas12a residues |

A rapid computational screen was then performed to test the ability of the HNH domain termini to connect to the two ends of the linker cut site using the Rosetta Remodel protocol (Huang P. S. et al 2011) included in the Rosetta macromolecular modeling software package. For each insertion point, ten iterations of loop closure (with no sequence design or insertions) were performed. The number of times that the linkers were able to successfully connect out of those ten iterations were tallied and compared (Table 1). Two of these insertion sites were then selected for more thorough linker modeling, including variations in linker lengths, based on a combination of their rate of successful loop closure and manual inspection (shown in bold in Table 1).

For the two selected insertion sites, fine-grained testing was then performed with small (2 to 4 residue) glycine-serine insertions or deletions in the N-terminal and C-terminal linkers and with more thorough sampling (100 iterations each). Possible residues for deletion were selected based on manual inspection of the sequence. Based on the linker modeling results, eight designs (four for each insertion site) were selected for experimental testing including extensions of 0, 2, or 4 residues of the N-terminal linker and extensions of 0 or 2 residues of the C-terminal linker.

### Example 2

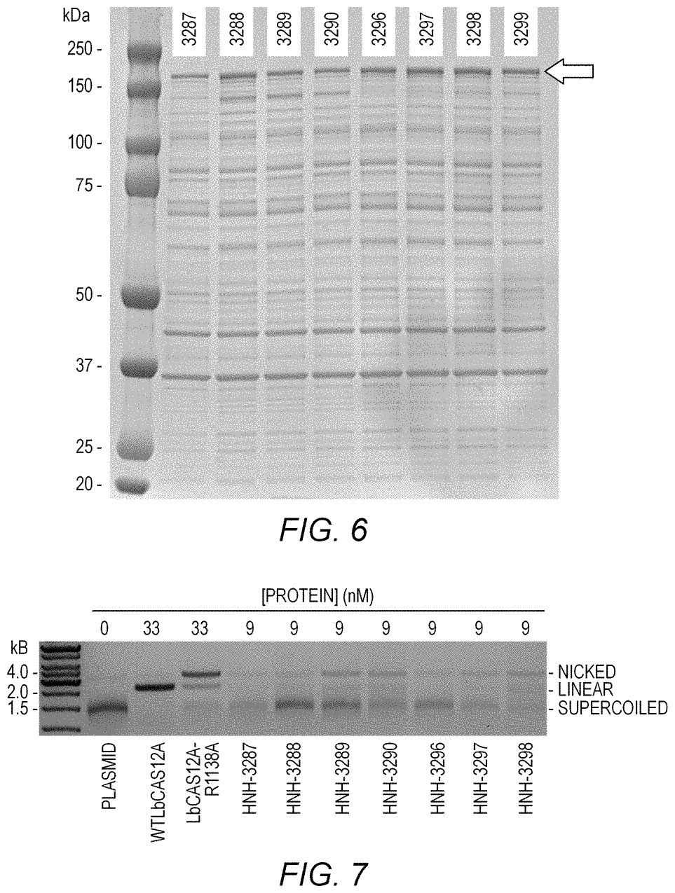

DNA coding regions for 8 LbCas12a-HNH constructs (HNH-3287, HNH-3288, HNH-3289, HNH-3290, HNH-3296, HNH-3297, HNH-3298, and HNH-3299) were synthesized with a 6-Histidine tag using solid state synthesis. The coding regions were coned into a pET28a plasmid (Novagen) behind an inducible T7 promoter and transfected into BL21(DE3)-Star cells (Invitrogen) and plated on kanamycin. Single colonies were grown in 30 ml of Luria Broth at 37° C. to an A600 optical density of 0.5. 500 mM IPTG was added and the temperature was lowered to 18° C. for 18 hours of expression. Cells were pelleted and lysed with BugBuster Master Mix (Millipore) according to the manufacturer's directions. Cell debris was pelleted and a soluble fraction was imaged on a 4-12% Bis-Tis PAGE gel (Invitrogen) under reducing conditions, and visualized using Coomassie staining. All eight HNH constructs showed soluble protein expression at the approximate MW of 160 kDa (FIG. 6, arrow).

Soluble protein expression for all eight constructs containing HNH nuclease in the middle of the Cas12a protein speaks to the quality of the fusion designs. Large domain insertions into the middle of proteins often results in insoluble protein expression or no expression in Escherichia coli. The observation of all eight proteins being highly expressed suggests the chimeric protein is folding properly and has not led to the disruption of either protein folds.

The expression protocol was repeated to generate proteins suitable for nuclease assays. After pelleting the eight constructs, the E. coli cells were frozen, thawed, and were suspended in Buffer A (20 mM HEPES-KOH pH 7.5, 500 mM NaCl, 10% glycerol, 2 mM TCEP, and 10 mM Imidazole pH 7.5). 0.3 mg/ml lysozyme was added, and the cells were incubated at room temperature for 20 minutes, followed by sonication (QSonica) with a ⅛-inch tip, 25% power, 10 second bursts followed by 30 second rests for 2.25 minutes. Cell debris was pelleted, and the supernatant was loaded onto Ni-NTA Agarose (Bio-Rad), washed with 20 mM imidazole in buffer A, and eluted with 300 mM imidazole in Buffer A. Approximate concentrations of proteins were 0.5 to 2 mg/ml (estimated by NanoDrop A280 absorbances) in a total eluate of 200 μL.

### Example 3

A plasmid-based assay was used to assess nicking activity by purified HNH-3287, HNH-3288, HNH-3289, HNH-3290, HNH-3296, HNH-3297, and HNH-3298. Plasmid nicking assays work on the principle that supercoiled plasmids extracted from bacteria run smaller on agarose gels than linearized, double-cut plasmid. Furthermore, if only one strand is nicked the plasmid runs even larger than linearized plasmid. This assay has been used extensively in the CRISPR field to assess if an enzyme is a double-stranded nuclease or a single-stranded nuclease (Jinek et al., Science. 2012 Aug. 17; 337(6096):816-21) (Zetsche et al., Cell. 2015 Oct. 22; 163(3):759-71).

The sequence 5′-TTTAGGAAT CCCTTCTGC AGCACCTGG-3′ (SEQ ID NO:123), where the protospacer-adjacent motif (PAM) is in bold, was synthesized and cloned into a pUC18 plasmid. The plasmid was expressed in DH5a cells and purified using plasmid miniprep kits (Qiagen). CRISPR RNA molecules were synthesized (Synthego) without any chemical modifications with the sequence 5′-AAUUUCUACU AAGUGUAGAU GGAAUCCCUU CUGCAGCACC UGG-3′ (SEQ ID NO:124) where the portion complimentary to the plasmid is emboldened. 30 μL reactions were assembled with a 10:10:1 RNA:Protein:Plasmid ratio, incubated for 15 minutes at 37° C., heat-inactivated at 85° C. for 2 minutes, and loaded on a 1% agarose gel containing 1/100 v/v SYBR-Safe stain (Invitrogen).

Proteins tested were wildtype LbCas12a (wtLbCas12a), LbCas12a-R1138A, and the various chimeric HNH proteins. The R1138A is a point mutation in LbCas12a which corresponds to a known non-template strand nickase mutation for AsCas12a (R1226A) (Yamano T, et al. Cell. 2016 May 5; 165(4):949-62). Concentrations tested were 33 nM for wtLbCas12a and LbCas12a-R1138. A lower 9 nM was used for the various HNH constructs to distinguish the most active nucleases by being somewhat near the expected Kd rather than generating complete nicking.

The resulting gel (FIG. 7) indicates that HNH-3287, HNH-3288, HNH-3289, HNH-3290, HNH-3296, HNH-3297, and HNH-3298 are all nickases with percentages of nicking from −25% efficiency to ˜75% efficiency (upper bands, the nicked plasmids, compared to lower bands, the supercoiled plasmids) at these low 9 nM protein concentrations. Longer incubations or higher concentrations result in complete nicking, but do not allow for comparing relative mutant activities. Chimera HNH-3298 appears to have the highest percentage of nicking activity with 9 nM [protein] for 15 minutes at 37° C.

### Example 4

### Methods

### Protein Expression and Purification

For initial testing of expression and activity, His-tagged proteins, SYN3287 (SEQ ID NO:125), SYN3288 (SEQ ID NO:126), SYN3289 (SEQ ID NO:127), SYN3290 (SEQ ID NO:128), SYN3296 (SEQ ID NO:129), SYN3297 (SEQ ID NO:130), SYN3298 (SEQ ID NO:131), and SYN3299 (SEQ ID NO:132), were expressed in BL21 cells in 30 mL cultures. Each of the proteins included an active HNH domain and an inactivated RuvC domain. Cells were pelleted, frozen overnight, and lysed by sonication. Proteins were then crudely purified from the lysate using HisPur™ Ni-NTA Spin Columns.

For assays of SYN3298 and SYN3289, proteins were expressed in the same way with the following changes: proteins were expressed in 1 L cultures and purified using HisTrap-HF columns by FPLC. Fractions containing the protein of interest were further purified by cation exchange and stored in 50% glycerol.

### Plasmid Nickase Assay

To determine the activity of purified proteins as nickases or nucleases, 30 μL reactions were prepared containing 1× NEBuffer 3.1, 100 femtomoles of the DNA substrate, and equal parts purified protein and an appropriate guide RNA (1 picomole of each unless otherwise noted). Reactions were incubated at 37° C. for 30 minutes, stopped by a 20 minute Proteinase K digestion at room temperature, and separated on a 1% agarose gel. The target site for the plasmid nickase assay had a sequence of SEQ ID NO:133.

### Fluorescent Nickase Assay

DNA substrates were produced by annealing one labeled (SEQ ID NO:134) and one unlabeled (SEQ ID NO:135) DNA strand to produce substrates labeled with Cy5 either on the PAM-containing or non-PAM-containing strand. The spacer for this assay included a sequence of SEQ ID NO:150. Nicking reactions were prepared as described for the plasmid nickase assay an incubated at 37° C. for 30 minutes. Reactions were stopped by digesting samples with Proteinase K for 10 minutes. All samples were then mixed with urea loading buffer to 1× concentration and heated to 90° C. for 5 minutes to denature the substrates. Samples were separated by running on a 6% TBE Urea gel at 4° C. and 100V.

### HEK293T Cell Transfection

Eukaryotic HEK293T (ATCC CRL-3216) cells were cultured in Dulbecco's Modified Eagle's Medium plus GlutaMax (ThermoFisher) supplemented with 10% (v/v) FBS (FBS), at 37° C. with 5% C02. Protein components were synthesized using gene synthesis and subsequently cloned into plasmids with a CMV promoter. Guide RNAs were cloned with a human U6 promoter. HEK293T cells were seeded on 48-well collagen-coated BioCoat plates (Corning). Cells were transfected at about 70% confluency. 375 ng of CRISPR plasmid and 125 ng of guide RNA expression plasmids were transfected using 1.5 μl of Lipofectamine 3000 (ThermoFisher Scientific) per well according to the manufacturer's protocol. Genomic DNA from transfected cells were obtained after 3 days and indels were detected and quantified using high-throughput Illumina amplicon sequencing.

To determine which strand the designed proteins preferentially nick, pairs of guides were designed such that a Cas9 guide and a guide for the designed proteins on the same strand would cut close to each other (within ˜10 bp). Each tested design was paired with either a nuclease-dead SpCas9, a SpCas9 D10A target strand nickase, or a SpCas9 H840A nontarget strand nickase. If the synthetic nickase and its paired Cas9 nickase cut opposite strands, then a greater editing frequency was expected than if they cut the same strand due to the production of double-stranded breaks.

### Results

His-Tagged Designed Synthetic Nickases were Successfully Expressed in BL21 E. coli.

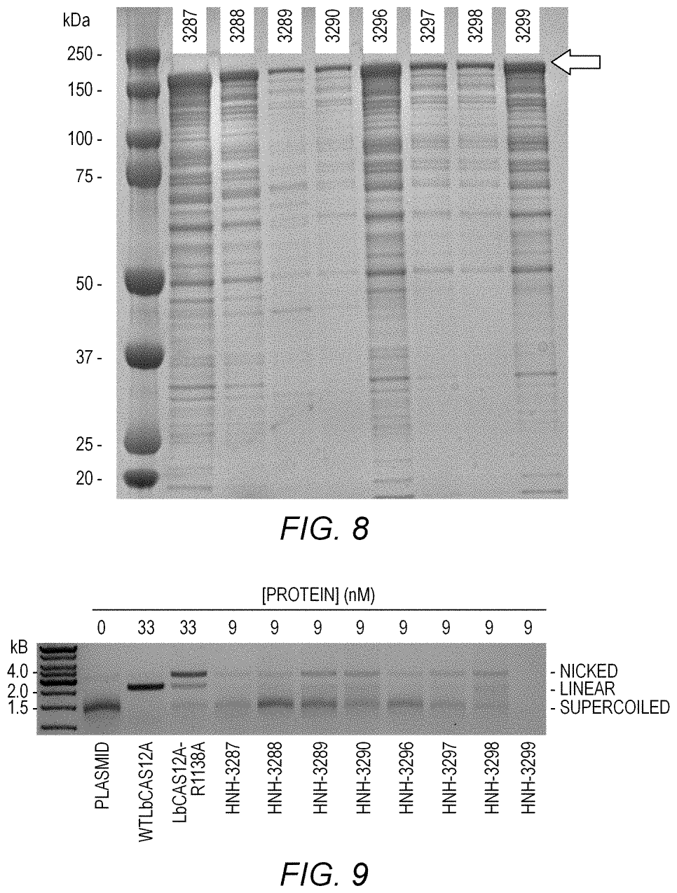

After crude purification of the designed nickases as described above, all samples showed a band at the expected size (˜160 kDa) as shown in FIG. 8, indicating that the nickases were solubly expressed in E. Coli.

Initial Plasmid Nicking Activity Observed from Crude Purifications of Synthetic Nickases.

Plasmid nickase assays were performed as described in the methods section above using the crude nickase purifications shown in FIG. 8. Due to low yields from some of the purifications, all designed nickases were tested at low concentrations so that they could be compared directly. As can be seen in FIG. 9, all but one of the designs showed a band indicating the presence of nicked plasmid that was more prominent than in the negative control sample, suggesting that the designs are capable of nicking a DNA substrate.

### RNA Dependence of Plasmid Nicking Using Crudely Purified Synthetic Nickases.

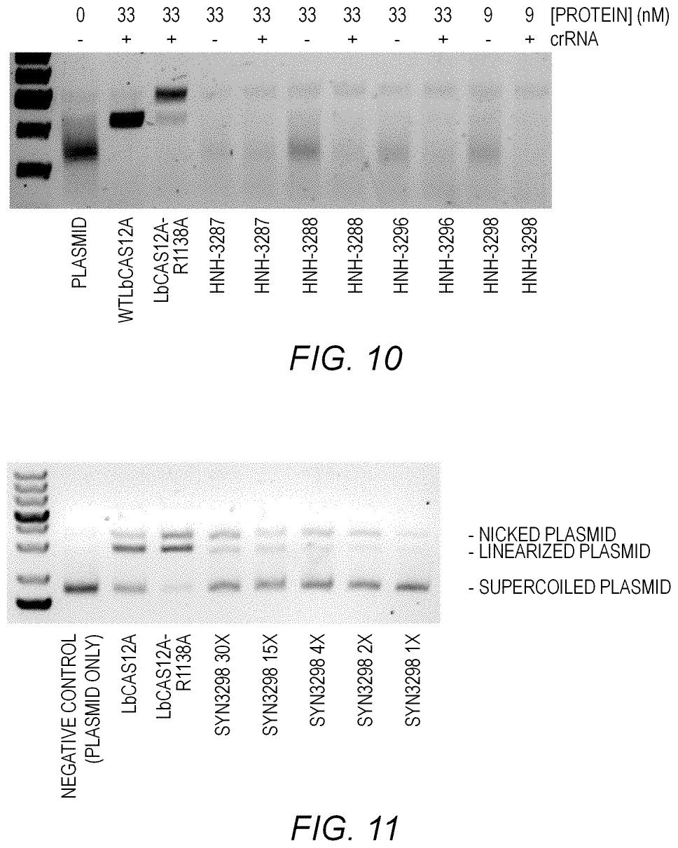

To ensure that the observed nicking and cleavage of the plasmid were guide-dependent and not due to random nuclease activity, the plasmid nickase assay was repeated for selected designs in both the presence of a targeting crRNA. Designs SYN3288, SYN3296, and SYN3298 all showed a reduction in the amount of uncleaved plasmid present in the presence of crRNA as can be seen in FIG. 10, indicating that their nuclease activity is RNA-dependent.

### Plasmid Nicking Activity of Purified Synthetic Nickase SYN3298.

Different quantities of protein+guide were tested relative to the concentration of LbCas12a control used (e.g. 30× indicates that 30 picomoles of protein and guide were included in the reaction). Nicking and, to a lesser extent, cleavage of the plasmid were observed at all tested concentrations of SYN3298 (FIG. 11), confirming that this design acts as a DNA nickase.

### Fluorescent Nickase Assay Using Purified Synthetic Nickases SYN3298 and SYN3289.

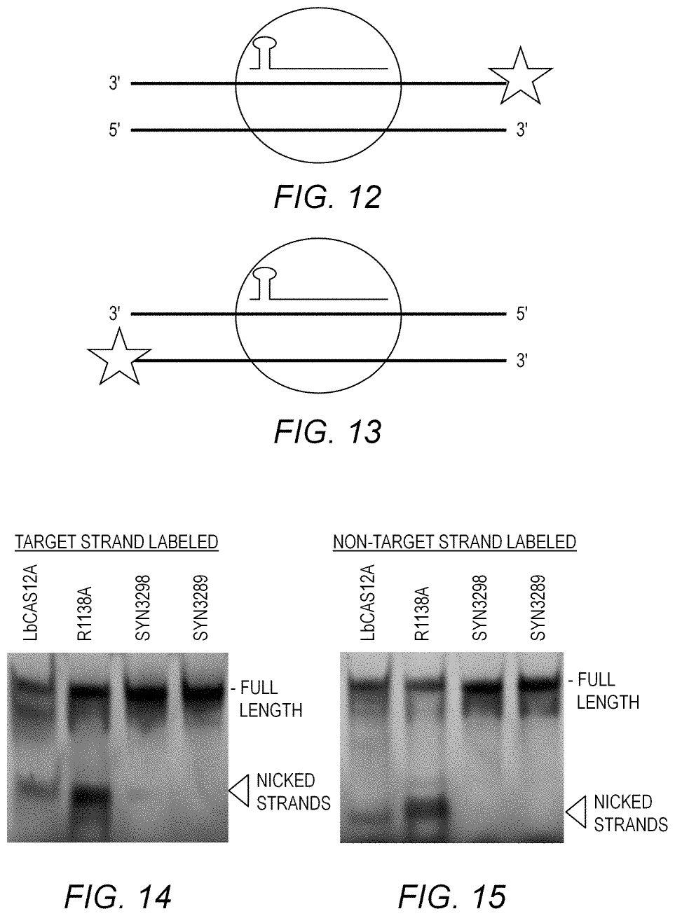

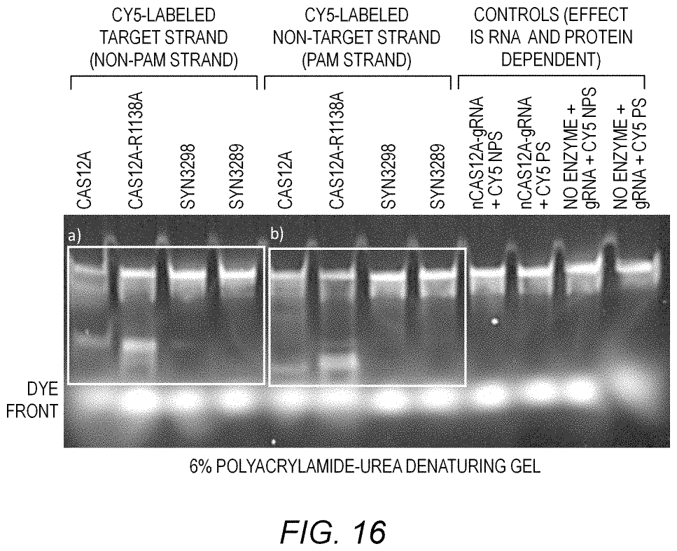

Substrates with a fluorescent Cy5 label on either the target (FIG. 12) or the nontarget (FIG. 13) were incubated with the designed nickases (which included an active HNH domain and an inactivated RuvC domain), LbCas12a, or the LbCas12a R1138a mutant (a nontarget strand nickase) and separated on a denaturing TBE-Urea gel. A shift in the position of the labeled band indicates that that strand was cleaved. FIG. 14 shows a portion of the gel for the samples incubated with the labeled target strand, FIG. 15 shows a portion of the gel for the samples incubated with the labeled non-target strand, and FIG. 16 shows the entire gel with lanes for the controls, the samples incubated with the labeled target strand (the boxed lanes denoted as “a)”), and the samples incubated with the labeled non-target strand (the boxed lanes denoted as “b)”). SYN3298 shows bands at the expected location for a cleaved substrate for the target DNA strand but not the nontarget DNA strand, indicating that it acts as a target strand nickase.

### Sequence-Based Strand-Specific Nickase Assay of Genomic DNA in HEK293T Cells.

Synthetic nickases were co-transfected with nearby Cas9 nickases (e.g., Cas9(H840A) or Cas9(D10A) cutting either the target strand (e.g., Cas9(D10A)) or nontarget strand (e.g., Cas9(H840A)). Information on the spacers used in the sequence based, strand specific nickase assay is provided in Table 2. Upstream guide refers to which spacer would be expected to cut closer to the 5′ end of the PAM-containing DNA strand. Estimated distance between the cut sites was determined based on the predicted cut site for each native nuclease domain.

TABLE 2
Spacer information for the sequence based, strand specific nickase assay.

| | | | Estimated | | |
|---|---|---|---|---|---|
| | | Cas12a/synthetic | distance | | |
| | Cas9 spacer | nickase | between cut | | Upstream |
| Target | sequence | spacer sequence | sites | | guide |
| RUNX1 | GCATTTTCAGGAG | CAGGAGGAAGCGATGG | 7-12 | bp | Cas9 |
| | GAAGCGA | CTTCAGA | | | |
| | (SEQ ID NO: 136) | (SEQ ID NO: 137) | | | |
| AAV1 | GTCCCCTCCACCC | TCTGTCCCCTCCACCCC | 2-7 | bp | Cas 12a/ |

| | | |
|---|---|---|
| CACAGTG | ACAGTG | synthetic |
| (SEQ ID NO: 138) | (SEQ ID NO: 139) | nuclease |

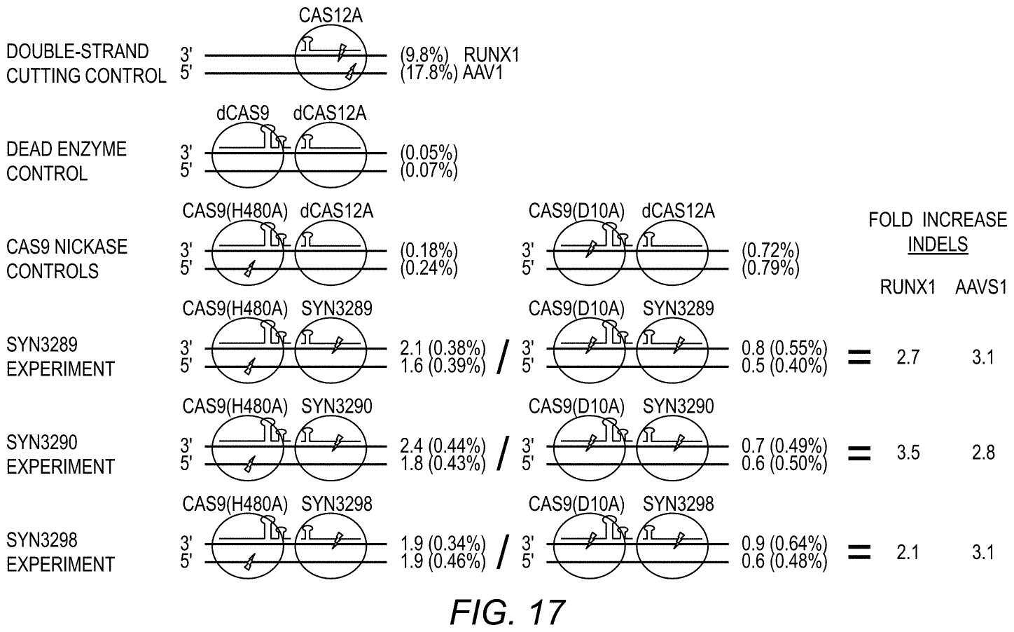

Editing efficiencies for each enzyme pair were normalized to the observed level of indels when the Cas9 nickase was paired with a nuclease-dead LbCas12a at the same target site (FIG. 17). Numbers in parentheses in FIG. 17 indicate the observed editing efficiencies prior to normalization. If the synthetic enzyme (SYN) (i.e., SYN3289, SYN3290, or SYN3298) preferentially cuts the target strand then (H480A::SYN)/(D10A::SYN)>1. If the synthetic enzyme (SYN) (i.e., SYN3289, SYN3290, or SYN3298) preferentially cuts the nontarget strand then (H480A::SYN)/(D10A::SYN)<1.

To determine which strand the designed proteins preferentially nick, pairs of guides were designed such that a Cas9 guide and a guide for the designed proteins on the same strand would cut close to each other (within ˜10 bp). Each tested design was paired with either a nuclease-dead SpCas9, a SpCas9 D10A target strand nickase, or a SpCas9 H840A nontarget strand nickase. If the synthetic nickase and its paired Cas9 nickase cut opposite strands, then a greater editing frequency was expected to be seen than if they cut the same strand due to the production of double-stranded breaks. Increased (about a 3-fold increase) indel frequency was consistently observed when all designed nickases were paired with a Cas9 nontarget strand nickase compared to a Cas9 target strand nickase, indicating that the designed nickases preferentially cut the target DNA strand.

### Example 5

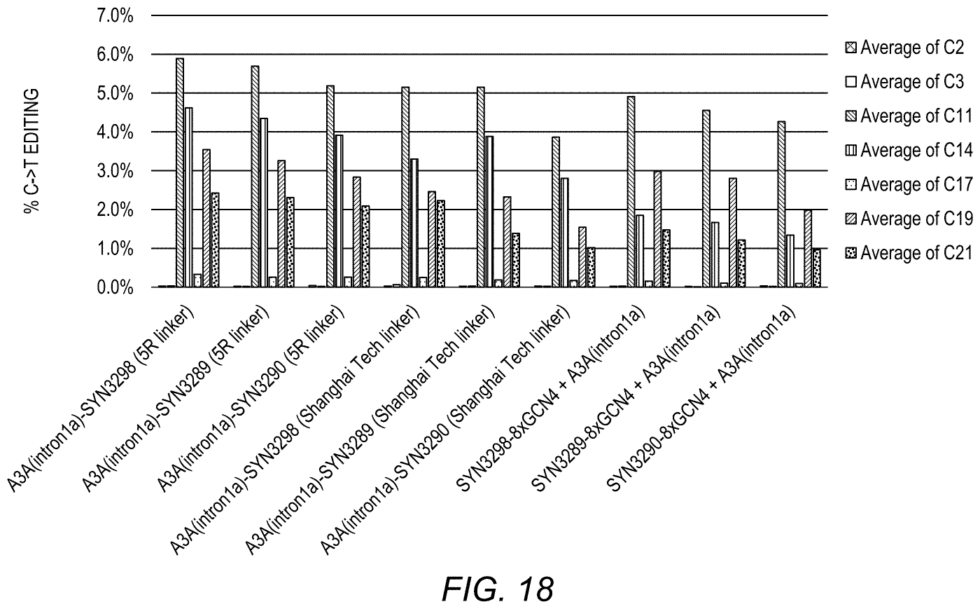

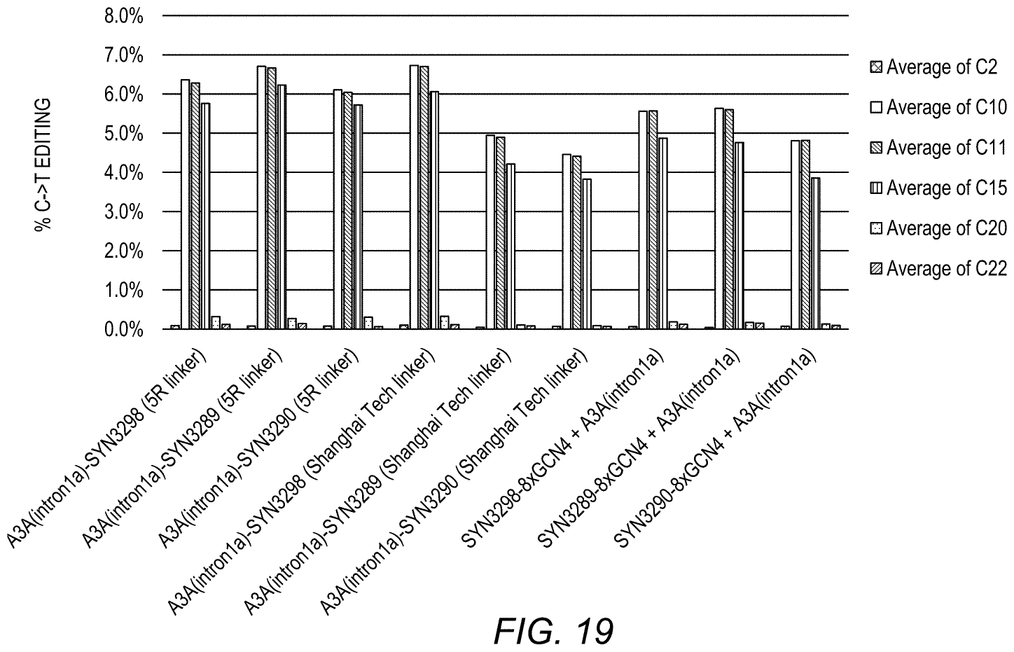

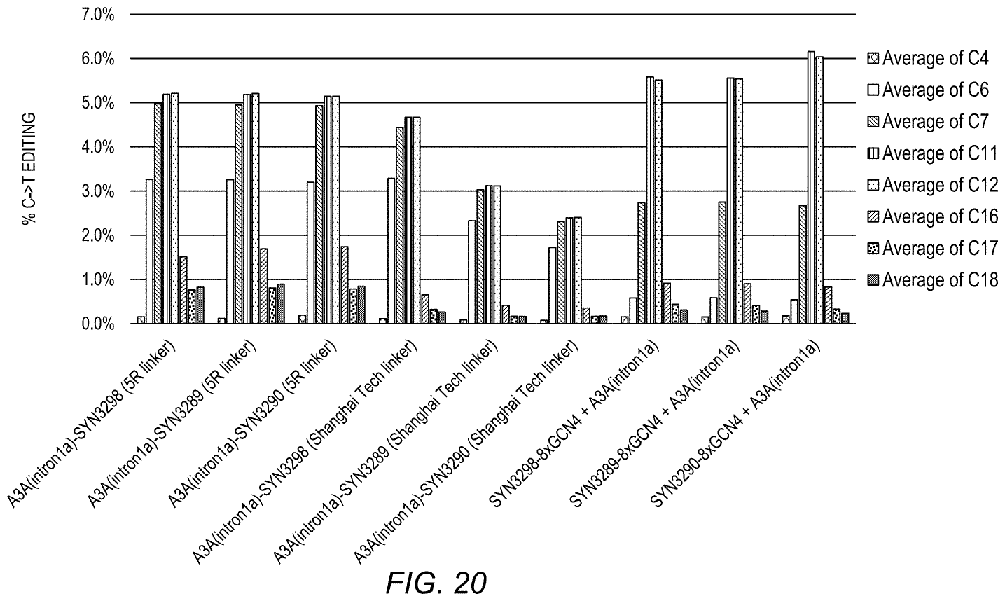

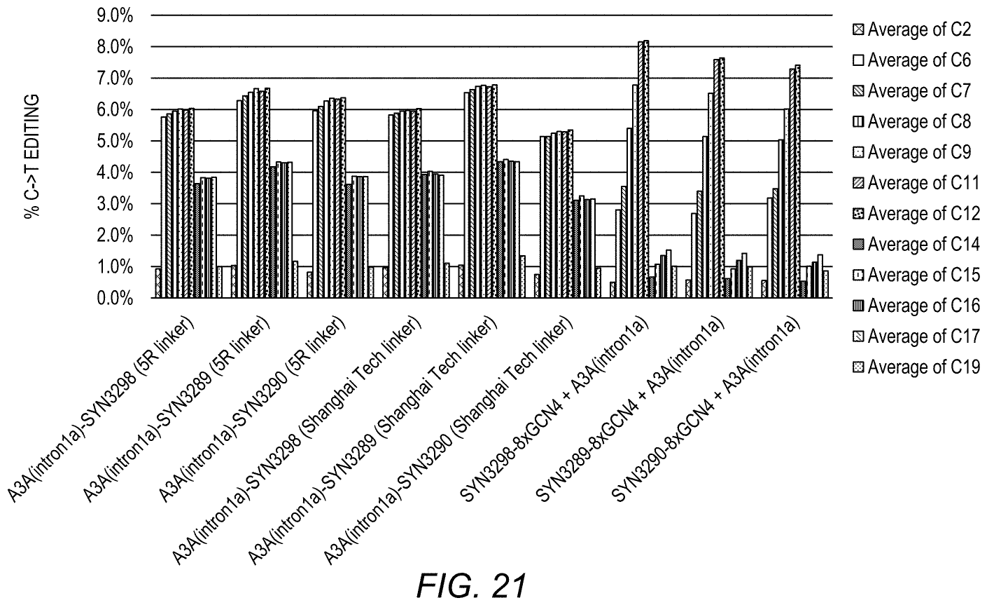

Cytosine base editing data for base editors combining the A3A cytosine deaminase (SEQ ID NO:152) with SYN3289, SYN3290, or SYN3298 was obtained (FIGS. 18-21). Three architectures were tested for each enzyme: Fusion of A3A to the N-terminus of the synthetic enzyme using a linker (SEQ ID NO:22) along with fusion of UGI (SEQ ID NO:104) to the C-terminus of the synthetic enzyme using a linker of SEQ ID NO:45 to provide SEQ ID NOs:160-162; fusion of A3A to the N terminus of the synthetic enzyme using a previously published linker (SEQ ID NO:153; Li et al. Nat Biotechnol 36, 324-327 (2018)) along with fusion of UGI (SEQ ID NO:104) to the C-terminus of the synthetic enzyme using a linker of SEQ ID NO:154 to provide SEQ ID NOs:163-165; or Suntag-based recruitment of UGI (SEQ ID NO:104) fused to the C-terminus of A3A (SEQ ID NO:152) to provide SEQ ID NO:156 recruited to the peptide tagged synthetic enzyme of one of SEQ ID NOs:157-159. All percentages shown in FIGS. 18-21 indicate averages across three data points. All of the tested enzymes demonstrated cytosine base editing in all three of the tested configurations. The spacer for FIG. 18 was SEQ ID NO:144, the spacer for FIG. 19 was SEQ ID NO:145, spacer for FIG. 20 was SEQ ID NO:146, and the spacer for FIG. 21 was SEQ ID NO:147.

### Example 6

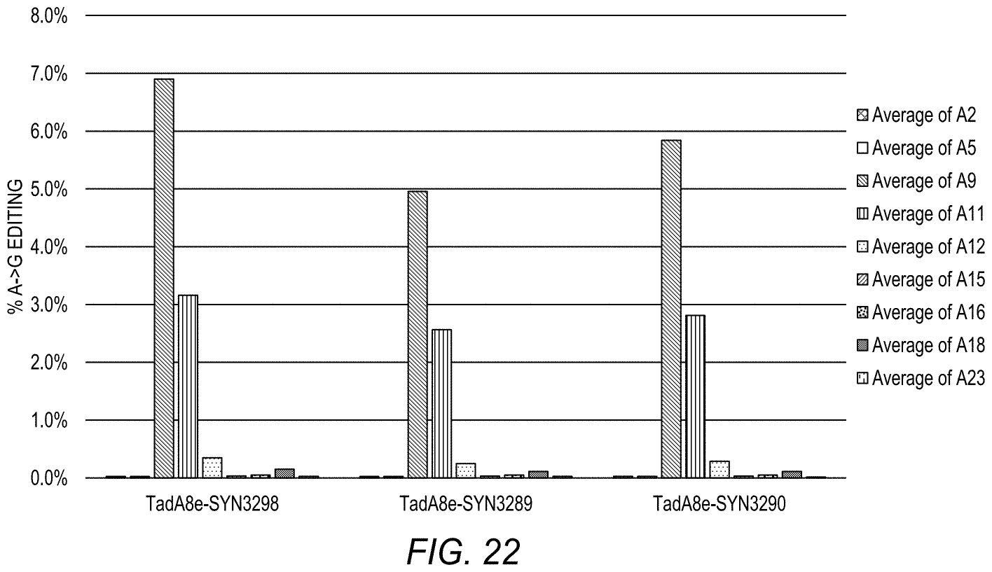

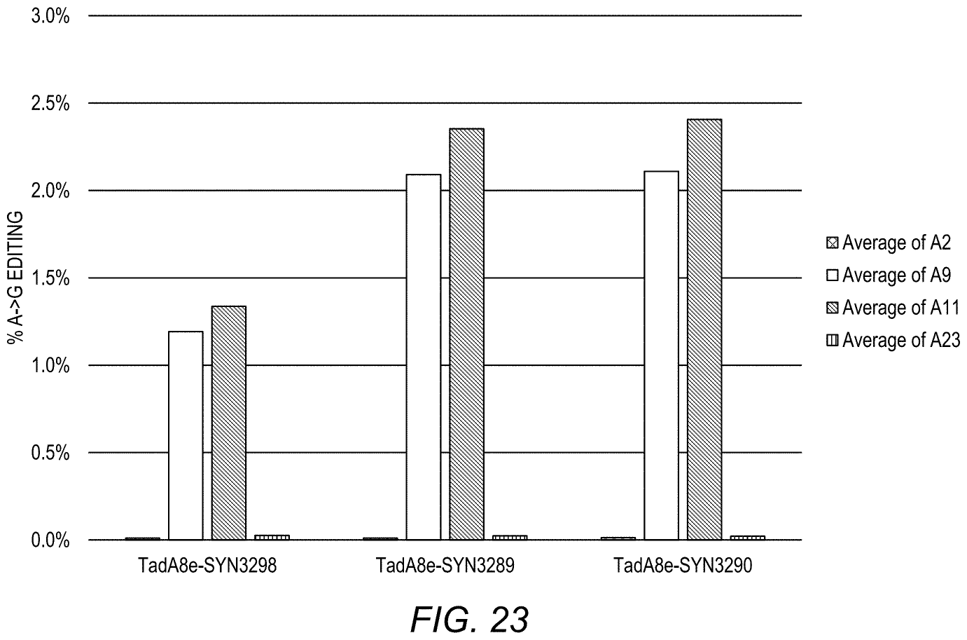

Adenine base editing data for synthetic enzymes SYN3289, SYN3290, and SYN3298 as N-terminal fusions to the TadA8e adenine deaminase was obtained (FIGS. 22-23). The synthetic enzymes were fused to TadA8e (SEQ ID NO:155) using a linker (SEQ ID NO:47) to provide SEQ ID NOs:166-168. All percentages shown in FIGS. 22-23 indicate averages across three data points. The three tested designs all demonstrated adenine base editing activity when fused with TadA8e. The spacer for FIG. 22 was SEQ ID NO:148 and the spacer for FIG. 23 was SEQ ID NO:149.

The foregoing is illustrative of the present invention, and is not to be construed as limiting thereof. The invention is defined by the following claims, with equivalents of the claims to be included therein.

1. An engineered Cas effector protein comprising at least two different polypeptides,
wherein one of the at least two different polypeptides is a first CRISPR-Cas effector polypeptide that is a first portion of a first Type V CRISPR-Cas effector protein and the first CRISPR-Cas effector polypeptide is devoid of a nuclease domain; and
wherein another of the least two different polypeptides is a heterologous polypeptide that is heterologous to the first Type V CRISPR-Cas effector protein and is not a portion of a Type V CRISPR-Cas effector protein.

2. (canceled)

3. The engineered Cas effector protein of claim 1, wherein the heterologous polypeptide comprises a first nuclease domain or a portion thereof that is heterologous to the first CRISPR-Cas effector polypeptide.

4. The engineered Cas effector protein of claim 1, wherein the heterologous polypeptide comprises a target strand nickase domain or a portion thereof.

5. The engineered Cas effector protein of claim 1, further comprising a second CRISPR-Cas effector polypeptide that comprises a second nuclease domain or a portion thereof.

6. The engineered Cas effector protein of claim 5, wherein the heterologous polypeptide is heterologous to the second CRISPR-Cas effector polypeptide.

7. The engineered Cas effector protein of claim 5, wherein the first and second CRISPR-Cas effector polypeptides are each a portion of the same CRISPR-Cas effector protein.

8-10. (canceled)

11. The engineered Cas effector protein of claim 1, wherein the heterologous polypeptide comprises a HNH domain.

12. The engineered Cas effector protein of claim 5, wherein the first and second CRISPR-Cas effector polypeptides are each a portion of a first CRISPR-Cas effector protein and the heterologous polypeptide is between and/or linked to two amino acids that are two consecutive or nonconsecutive amino acids of the first CRISPR-Cas effector protein.

13. The engineered Cas effector protein of claim 12, wherein the heterologous polypeptide is positioned in the engineered Cas effector protein in a location that corresponds to an interdomain linker region of the first CRISPR-Cas effector protein.

14. The engineered Cas effector protein of claim 1, wherein the heterologous polypeptide comprises an amino acid sequence having at least 70%, 75%, 80%, 85%, 90%, 95%, 96%, 97%, 98%, 99%, or more sequence identity to one or more of SEQ ID NOs:1 or 169-174.

15. (canceled)

16. The engineered Cas effector protein of claim 1, further comprising all or a portion of a wedge domain, a Rec1 domain, a Rec2 domain, a PAM-interacting domain, a RuvC domain, a bridge helix, and/or a Nuc domain of the first Type V CRISPR-Cas effector protein.

17. The engineered Cas effector protein of claim 16, wherein the engineered Cas effector protein comprises the Rec domain and the Rec2 domain and the heterologous polypeptide is between the Rec1 domain and the Rec2 domain.

18.-21. (canceled)

22. The engineered Cas effector protein of claim 1, wherein the engineered Cas effector protein comprises an amino acid sequence having about 70%, 75%, 80%, 85%, 90%, 95%, 96%, 97%, 98%, 99%, or more sequence identity to the amino acid sequence of a wild-type CRISPR-Cas effector protein.

23. The engineered Cas effector protein of claim 1, wherein the engineered Cas effector protein comprises an amino acid sequence having about 70%, 75%, 80%, 85%, 90%, 95%, 96%, 97%, 98%, 99%, or more sequence identity to any one of SEQ ID NOs:2-17, 125-132, or 157-168.

24.-46. (canceled)

47. An engineered Cas effector protein comprising:
a first polypeptide that is a first portion of a first Type V CRISPR-Cas effector protein;
a second polypeptide that is a second portion of the first Type V CRISPR-Cas effector protein; and
a heterologous polypeptide that is heterologous to the first Type V CRISPR-Cas effector protein,
wherein the heterologous polypeptide is between the first and second polypeptides and the heterologous polypeptide is positioned in the engineered Cas effector protein in a location that corresponds to an interdomain linker region of the first Type V CRISPR-Cas effector protein.

48. The engineered Cas effector protein of claim 47, wherein the interdomain linker region comprises one or more amino acids of amino acid residues 283-293 of SEQ ID NO:50 or of corresponding amino acid residues for a sequence that is optimally aligned to SEQ ID NO:50.

49. The engineered Cas effector protein of claim 47, wherein first polypeptide comprises all or a portion of a wedge domain and/or a Rec1 domain of the first Type V CRISPR-Cas effector protein; and/or
the second polypeptide comprises all or a portion of a wedge domain, a Rec2 domain, a PAM-interacting domain, a RuvC domain, a bridge helix, and/or a Nuc domain of the first Type V CRISPR-Cas effector protein.

50. The engineered Cas effector protein of claim 49, wherein the engineered Cas effector protein comprises the Rec1 domain and the Rec2 domain and the heterologous polypeptide is between the Rec1 domain and the Rec2 domain.

51. (canceled)

52. The engineered Cas effector protein of claim 47, wherein the heterologous polypeptide comprises a target strand nickase domain or a portion thereof.

53. The engineered Cas effector protein of claim 47, wherein the second polypeptide comprises a nuclease domain or a portion thereof.

54.-74. (canceled)
